(19) United States
(12) Patent Application Publication
(10) Pub. No.: US 2010/0022574 A1
(43) Pub. Date: Jan. 28, 2010

(54) Methods to Treat Pain Using an Alpha-2 Adrenergic Agonist and an Endothelin Antagonist

(75) Inventor: Anil Gulati, Naperville, IL (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: EndogenX, Inc., Los Gatos, CA (US)

(21) Appl. No.: 12/180,829

(22) Filed: Jul. 28, 2008

(51) Int. Cl. A61K 31/42; A61K 31/4168; A61K 31/485; A61P 25/04

(52) U.S. Cl. 514/282; 514/401; 514/380

(57) ABSTRACT

The present invention relates, in general to treatment of pain comprising administering an alpha-2 adrenergic agonist and an endothelin antagonist, wherein administration of the agents acts as an analgesic and ameliorates pain in a subject.

### FIELD OF THE INVENTION

The present invention is related in general to treatment of pain and enhancement of analgesia comprising administering to a subject a combination of an alpha-2 adrenergic agonist with or without imidazoline activity and an endothelin receptor antagonist.

### BACKGROUND OF THE INVENTION

Analgesics are agents that relieve pain by acting centrally to elevate pain threshold, preferably without disturbing consciousness or altering other sensory functions. A mechanism by which analgesic drugs obtund pain (i.e., raise the pain threshold) has been formulated.

National Center for Health Statistics (2006) estimates more than one-quarter of Americans (26%) over the age of 20 years, and more than 76.5 million Americans, report that they have had a problem with pain of any sort that persisted for more than 24 hours in duration and over 191 million acute pain events occurred in the United States. Opioids are the most commonly used analgesics for the clinical management of acute and chronic pain. There are various side effects associated with the long-term use of opioids including the development of tolerance, which results in inadequate pain relief. There are several existing regimens designed to enhance analgesia and effectively manage pain, including nonsteroidal anti-inflammatory drugs (NSAIDS), additional opioids, and non-opioids in combination with opioid therapy. Although these approaches provide symptomatic relief, they have little effect on the underlying mechanisms that contribute to the development of tolerance and pose a significant risk of toxicity, dependence, and addiction.

Clonidine, an alpha-2 (α₂) adrenergic agonist, has been demonstrated to produce significant analgesia in mice and rats [31 ] and it was also found that repeated administration of clonidine (twice daily for 7 days) produced tolerance, whereas, acute administration of clonidine enhanced the analgesic effect of morphine [30]. Clonidine potentiated morphine analgesia but there was no cross tolerance between clonidine and morphine analgesia [38]. It has been proposed that clonidine may be responsible for the activation of adrenergic and opiate antinociceptive mechanism in the diencephalic periventricular gray, the dorsal raphe nuclei, and the periaqueductal gray [39]. Clonidine has also been used to suppress opiate withdrawal. These two properties theoretically make it a suitable analgesic substitute in patients tolerant to opioids [27]. Clonidine has been demonstrated to enhance not only morphine analgesia but also enhances analgesic actions of pentazocine [13], fentanyl [9], and bupivacaine [19].

The endothelins (ETs) constitute a family of endothelium-derived polypeptides that are among the most potent vasoconstrictors known. The major endothelin receptor subtypes (ET_{A} and ET_{B}) are expressed in vascular smooth muscle, where they mediate vasoconstriction. The ET_{B} subtype on endothelial cells is believed to meditate vasorelaxation. ET_{A} receptor antagonists have been found to significantly potentiate the antinociceptive response to morphine in both rats and mice [5,7,16]. Chronic injections of the ET_{A} receptor antagonist BQ123 along with morphine prevented the development of tolerance to morphine [6]. A single injection of ETA receptor antagonists was found to reverse the tolerance to morphine analgesia in mice and rats [4,6,7]. Therefore, the phenomenon of tolerance was prevented and reversed by ET_{A} receptor antagonists demonstrating that ET is involved in morphine tolerance [6,7,33,34]. Furthermore, ET_{A} antagonists did not have any effect on side effects of morphine such as cataleptic action [5] or gastrointestinal transit [26]. Therefore, ET_{A} receptor antagonists potentiate morphine analgesia without enhancing side effects of morphine [16].

Sulfisoxazole is commonly used for the treatment of otitis media and for the treatment of bacterial infections, and has excellent antibacterial activity. The chemical structure of sulfisoxazole is 4-amino-N-(3,4-dimethyloxazol-5-yl)-benzenesulfonamide. Sulfisoxazole is highly soluble. When administered orally, sulfisoxazole is rapidly absorbed and rapidly excreted and is highly soluble and is therefore reduces the renal toxicity inherent in the use of sulfonamides [25]. The inhibitory effect of the sulfanilamides on the binding of ET-1 to ET_{A} and ET_{B} receptors was determined in membrane preparations. Sulfisoxazole was the most active sulfanilamide with an IC50 of 0.60 μM and 22 μM for ET_{A} and ET_{B} receptors, respectively [8].

An interaction between clonidine and ET in the cardiovascular effects involving sympathetic nervous system has been reported [15,17,18,28]. Endothelin-1 (ET-1) has also been found to potentiate hypotension produced by clonidine. This effect may be mediated through nitric oxide mechanism [23]. No study has been performed to determine the interaction of clonidine and ET_{A} receptor antagonists on analgesia.

Thus there exists a need in the art to identify agents, or combinations of agents that reduce tolerance to opioid pain relievers and reduce pain symptoms, and that can act as effective non-opioid analgesics.

### SUMMARY OF THE INVENTION

The present invention relates to the use of an adrenergic agonist and an endothelin antagonist as an analgesic to treat pain in a subject. Additionally, it has been discovered herein that an alpha 2 (α2) adrenergic agonist in combination with an endothelin A (ET_{A}) antagonist can potentiate the analgesic effects of opioid analgesics, as well as act in combination to produce analgesia. In one aspect, the invention provides a method of treating or preventing pain comprising administering to a mammal in need thereof a therapeutically effective amount of an alpha-2 (α₂) adrenergic receptor agonist and a therapeutically effective amount of an endothelin receptor antagonist.

In one embodiment the α₂ adrenergic agonist is an agonist with or without imidazoline activity. In a further embodiment, the α₂ adrenergic agonist is selected from the group consisting of dexmedetomidine, detomidine, ST-91, medetomidine, brimonidine, tizanidine, mivazerol, guanabenz, guanfacine, iodoclonidine, xylazine, rilmenidine, lofexidine, azepexole, alpha-methyldopa, and alpha-methylnoradrenaline or a derivative, salt or structural analogue thereof. In a related embodiment, the α₂ adrenergic agonist is clonidine.

In a further embodiment, it is contemplated that the endothelin receptor antagonist is an endothelin receptor A (ET_{A}) antagonist. In another embodiment, the ET_{A} antagonist is selected from the group consisting of sulfosoxazole, atrasentan, tezosentan, bosentan, sitaxsentan, enrasentan, BMS 207940, BMS 193884, BMS 182874, J 104132, VML 588/Ro 61 1790, T-0115, TAK 044, BQ 788, TBC2576, TBC3214, PD180988, ABT 546, SB247083, RPR118031A and BQ123. Instill another embodiment, the ET_{A} antagonist is sulfisoxazole.

In one aspect, the α₂ adrenergic agonist and the endothelin receptor antagonist are administered in a single composition. In a related aspect, the α₂ adrenergic agonist and the endothelin receptor antagonist are administered in separate compositions. In one embodiment, the compositions are administered concurrently. In a related embodiment, the compositions are administered separately. In a further embodiment, the α₂ adrenergic agonist and endothelin receptor antagonist molecules of the combination are administered sequentially within about a 24-hour period.

In a further embodiment, the compositions further comprise a pharmaceutical carrier or excipient.

In yet another embodiment, the α₂ adrenergic agonist and the endothelin receptor antagonist are administered orally, buccally, via inhalation, sublingually, rectally, vaginally, intracisternally, intraarticularly, transurethrally, nasally, percutaneously, intravenously, intramuscularly, or subcutaneously.

In one embodiment, the clonidine is administered in a dose range from about 10 μg to about 300 μg. In a related embodiment, the sulfisoxazole is administered in a dose range from about 0.1 g to about 3 g. It is further contemplated that the ratio of α₂ adrenergic agonist administered to endothelin receptor antagonist administered is in the range of 1:500 to 1:50,000, 1:500 to 1:20,000, 1:500 to 1: 10,000, 1:500 to 1:5,000, 1:500 to 1:2,500, 1: I 00 to 1: I 000, or 1: I 00 to 1:500.

In another aspect, the invention provides a method of treating or preventing pain comprising administering to a subject a synergistic combination of one or more alpha-2 (α₂) adrenergic agonist and one or more endothelin receptor antagonist. In one embodiment, the agonist and antagonist molecules of the combination are administered sequentially within about a 24-hour period or are administered concurrently. It is further contemplated that administration of the agents occurs within the range of 30 minutes up to about one day (24 hours).

The invention further provides a composition for treating or preventing pain comprising a synergistic combination of one or more alpha-2 (α₂) adrenergic agonist and one or more endothelin receptor antagonist. In one embodiment, the composition comprises a low dose of the α₂ adrenergic agonist and a low dose of the endothelin receptor antagonist. In a related embodiment, the α₂ adrenergic agonist is clonidine and the endothelin receptor antagonist is sulfisoxazole. In still a further embodiment, it iscontemaplted that the clonidine in the composition is in a range of about 10 μg to about 300 μg and the sulfisoxazole in the composition is in a range of about 0.1 g to about 3 g.

In yet another embodiment, the composition further comprises a pharmaceutically acceptable carrier.

In a further aspect, the invention provides for use of a composition comprising an alpha-2 (α₂) adrenergic agonist and an endothelin receptor antagonist for the manufacture of a medicament for treating pain in a subject.

The invention contemplates that the subject to be treated is a mammal. In one embodiment, the mammalian subject is human, or any non-human animal model for human medical research, or an animal of importance as livestock or pets, for example, companion animals. In a related embodiment, the subject is a human.

In one embodiment, the pain to be treated is chronic pain or acute pain. In a related embodiment, the pain is selected from the group consisting of causalgia, tactile allodynia, neuropathic pain, hyperalgesia, hyperpathia, inflammatory pain, post-operative pain, chronic lower back pain, cluster headaches, postherpetic neuralgia, phantom limb and stump pain, central pain, dental pain, neuropathic pain, opioid-resistant pain, visceral pain, surgical pain, bone injury pain, diabetic neuropathy pain, post-surgery or traumatic neuropathy pain, peripheral neuropathy pain, entrapment neuropathy pain, neuropathy caused by alcohol abuse, pain from HIV infection, multiple sclerosis hypothyroidism or anticancer chemotherapy pain, pain during labor and delivery, pain resulting from burns, including sunburn, post partum pain, migraine, angina pain, and genitourinary tract-related pain including cystitis.

In a further aspect, one or more alpha-2 (α₂) adrenergic agonist or one more endothelin receptor antagonist are useful to potentiate the analgesic effects of an opiate analgesic. In one embodiment, the combination of one or more alpha-2 (α₂) adrenergic agonist and one or more endothelin antagonist is useful to potentiate the analgesic effect of an opiate analgesic. As such, the invention provides a method of treating or preventing pain comprising administering to a mammal in need thereof a therapeutically effective amount of an opiate analgesic, and a therapeutically effective amount of a composition comprising one or one or more alpha-2 (α₂) adrenergic agonist. In a related embodiment, the invention contemplates s method of treating or preventing pain comprising administering to a mammal in need thereof a therapeutically effective amount of an opiate analgesic, a therapeutically effective amount of a composition comprising one or one or more alpha-2 (α₂) adrenergic agonist, and a therapeutically effective amount of a composition comprising one or more endothelin receptor antagonist. In a further embodiment, the invention provides a method of treating or preventing pain comprising administering to a mammal in need thereof a therapeutically effective amount of an opiate analgesic, and a therapeutically effective amount of a composition comprising one or one or more alpha-2 (α₂) adrenergic agonist and one or more an endothelin receptor antagonist.

In one embodiment the one or more alpha-2 (α₂) adrenergic agonist is selected from the group consisting of dexmedetomidine, detomidine, ST-91, medetomidine, brimonidine, tizanidine, mivazerol, guanabenz, guanfacine, iodoclonidine, xylazine, rilmenidine, lofexidine, azepexole, alpha-methyldopa, and alpha-methylnoradrenaline or a derivative, salt or structural analogue thereof. In a related embodiment, the one or more endothelin antagonists is an ET_{A} selected from the group consisting of sulfosoxazole, atrasentan, tezosentan, bosentan, sitaxsentan, enrasentan, BMS 207940, BMS 193884, BMS 182874, J 104132, VML 588/Ro 61 1790, T-0115, TAK 044, BQ 788, TBC2576, TBC3214, PD180988, ABT 546, SB247083, RPR1 18031A and BQ123.

In a further embodiment, the opiate analgesic is selected from the group consisting of morphine, morphine sulfate, codeine, diacetylmorphine; dextromethorphan, hydrocodone, hydromorphone, hydromorphone, levorphanol, oxymorphone, oxycodone, levallorphan and salts thereof.

In an embodiment, the opiate analgesic and one or more alpha-2 (α₂) adrenergic agonist and/or one or more an endothelin antagonist are administered simultaneously. In a related embodiment, the opiate analgesic and one or more alpha-2 (α₂) adrenergic agonist and/or one or more an endothelin antagonist are administered from a single composition or from separate compositions. In a further embodiment, the opiate analgesic and one or more alpha-2 (α₂) adrenergic agonist and/or one or more an endothelin antagonist are administered sequentially.

In yet another embodiment, the opiate analgesic is administered prior to the one or more alpha-2 (α₂) adrenergic agonist and/or one or more an endothelin antagonist or subsequent to the one or more alpha-2 (α₂) adrenergic agonist and/or one or more an endothelin antagonist.

It is provided that the dose of active ingredient described herein and administration regimens described herein are useful for all contemplated methods of the invention.

In another aspect, the invention contemplates an article of manufacture comprising an alpha-2 (α₂) adrenergic agonist and an endothelin receptor antagonist and a label indicating a method according to the present invention.

In yet another aspect, the invention provides a kit for treating or preventing pain comprising a composition comprising an alpha-2 (α₂) adrenergic agonist and an endothelin receptor antagonist; and a protocol for using the kit to treat pain. In one embodiment, the composition further comprise a pharmaceutically acceptable carrier. In a related embodiment, the kit further comprises an opiate analgesic. In a related embodiment, the opiate analgesic is in pharmaceutically acceptable carrier or excipient.

### BRIEF DESCRIPTION OF THE DRAWINGS

FIG. 1 shows the effect of clonidine (2 mg/kg, i.p.) on the tail flick latency in presence and absence of morphine (4 mg/kg, i.p.) Mice were divided in to four groups: group 1 received vehicle (saline i.p.)+vehicle (saline s.c.); group 2 received vehicle+morphine (4 mg/kg, s.c.); group 3 received clonidine (2 mg/kg, i.p.)+vehicle (saline s.c.); and group 4 received clonidine (2 mg/kg, i.p.)+morphine (4 mg/kg, s.c.). Morphine or vehicle was administered 30 min after clonidine administration. FIG. 1A shows the tail flick latency data in seconds at various time intervals. FIG. 1B shows antinociception as depicted by AUC_{0→240 min} determined from tail flick latency values. Values are mean ±SEM; N=6 per group. *P<0.05 clonidine compared to control group. ^{#}P<0.05 clonidine+morphine compared to vehicle+morphine group.

FIG. 2 shows the effect of sulfisoxazole (500 mg/kg, oral) on the tail flick latency in presence and absence of morphine (4 mg/kg, s.c.). Mice were divided in to four groups: group 1 received vehicle (oral carboxymethyl cellulose (CMC))+vehicle (saline s.c.); group 2 received vehicle (CMC)+morphine (4 mg/kg, s.c.); group 3 received sulfisoxazole (500 mg/kg, oral)+vehicle (saline s.c.); and group 4 received sulfisoxazole (500 mg/kg, oral)+morphine (4 mg/kg, s.c.). Morphine or vehicle was administered 30 min after sulfisoxazole administration. FIG. 2A shows the tail flick latency data in seconds at various time intervals. FIG. 2B shows antinociception as depicted by AUC_{0→240 min} determined from tail flick latency values. Values are mean ±SEM; N=6 per group. *P<0.05 sulfisoxazole compared to control group.

FIG. 3 shows the effect of clonidine (C) (2 mg/kg, i.p.) plus sulfisoxazole (S) (500 mg/kg, oral) on the tail flick latency in presence and absence of morphine (M) (8 mg/kg, s.c.). Mice were divided in to three groups: group 1 received clonidine (2 mg/kg, i.p.) plus sulfisoxazole (500 mg/kg, oral)+vehicle (V) (saline s.c.); group 2 received clonidine (2 mg/kg, i.p.) plus sulfisoxazole (500 mg/kg, oral)+morphine (8 mg/kg, s.c.); group 3 received vehicle (saline, i.p.) plus vehicle (carboxymethyl cellulose, oral)+morphine (M) (8 mg/kg, s.c.) and group 4 received vehicle (saline, i.p.) plus vehicle (carboxymethyl cellulose, oral)+vehicle (saline, s.c.). Morphine or vehicle was administered 30 min after clonidine plus sulfisoxazole administration. It was found that a combination of clonidine (2 mg/kg, i.p.) plus sulfisoxazole produced a significant increase in antinociception. Values are mean ±SEM; N=6 per group. *P<0.05 clonidine plus sulfisoxazole compared to control (vehicle) group.

FIG. 4 shows the effect of clonidine (2 mg/kg, i.p.) plus sulfisoxazole (1000 mg/kg, oral) on the antinociception as depicted by AUC_{0→240 min} determined from tail flick latency in the presence and absence of morphine (8 mg/kg, i.p.). Mice were divided in to six groups: group 1 received vehicle (saline i.p.) plus vehicle (carboxymethyl cellulose (CMC))+vehicle (saline s.c.): group 2 received clonidine (2 mg/kg, i.p.) plus vehicle (CMC)+vehicle (saline s.c.); group 3 received sulfisoxazole (1000 mg kg, oral) plus vehicle (saline i.p.)+vehicle (saline s.c.); group 4 received vehicle (saline i.p.) plus vehicle (CMC)+morphine (8 mg/kg s.c.); group 5 received clonidine (2 mg/kg, i.p.) plus sulfisoxazole (1000 mg kg, oral)+vehicle (saline s.c.); and group 6 received clonidine (2 mg kg, i.p.) plus sulfisoxazole (1000 mg/kg, oral)+morphine (8 mg/kg, s.c.). Morphine or vehicle was administered 30 min after clonidine plus sulfisoxazole administration. It was found that a combination of clonidine plus sulfisoxazole produced a significant increase in antinociception. Values are mean ±SEM; N=6 per group. *P<0.05 morphine compared to vehicle group. ^{#}P<0.05 clonidine plus sulfisoxazole compared to clonidine alone or sulfisoxazole alone groups.

FIG. 5 illustrates the dose response effect of clonidine on analgesia (tail flick latency) (FIG. 5A) and body temperature (FIG. 8B) in mice: Group 1: Vehicle (saline i.p.); Group 2: Clonidine (0.3 mg/kg, i.p.) ; Group 3: Clonidine (1.0 mg/kg, i.p.); Group 4: Clonidine (3.0 mg/kg, i.p.).

FIG. 6 shows the effect of clonidine plus sulfisoxazole on analgesia (FIG. 6A) and body temperature (FIG. 6B) in mice: Group 1: 0.5% carboxymethyl cellulose (CMC, p.o.)+Vehicle (saline i.p.); Group 2 CMC (p.o.)+Clonidine (0.3 mg/kg, i.p.); Group 3: sulfisoxazole (250 mg/kg, p.o.)+Clonidine (0.3 mg/kg, i.p.); Group 4: sulfisoxazole (500 mg/kg, p.o.)+Clonidine (0.3 mg/kg, i.p.); Group 5: sulfisoxazole (1000 mg/kg, p.o.)+Clonidine (0.3 mg/kg, i.p.).

FIG. 7 shows the effect of clonidine plus sulfisoxazole on analgesia (FIG. 7A) and body temperature (FIG. 7B) in mice: Group 1: 0.5% carboxymethyl cellulose (CMC, p.o.)+Vehicle (saline i.p.); Group 2 CMC (p.o.)+Clonidine (0.3 mg/kg, i.p.); Group 3: sulfisoxazole (25 mg/kg, p.o.)+Clonidine (0.3 mg/kg, i.p.); Group 4: sulfisoxazole (75 mg/kg, p.o.)+Clonidine (0.3 mg/kg, i.p.); Group 5: sulfisoxazole (225 mg/kg, p.o.)+Clonidine (0.3 mg/kg, i.p.).

FIG. 8 shows the effect of yohimbine on clonidine and clonidine plus sulfisoxazole on analgesia (FIG. 8A) and body temperature (FIG. 8B) in mice: Group 1: Vehicle+vehicle (saline i.p.)+vehicle (saline, i.p.); Group 2 Vehicle+CMC (p.o.)+vehicle (saline, i.p.); Group 3: Yohimbine (2 mg/kg, ip)+CMC (p.o.)+vehicle (saline, i.p.); Group 4: Yohimbine (2 mg/kg, ip)+CMC (p.o.)+Clonidine (0.3 mg kg, i.p.); Group 5: Yohimbine (2 mg/kg, ip)+sulfisoxazole (250 mg/kg, p.o.)+Clonidine (0.3 mg/kg, i.p.).

FIG. 9 shows the effect of idazoxan on clonidine and clonidine plus sulfisoxazole on analgesia (FIG. 9A) and body temperature (FIG. 9B) in mice: Group 1: Vehicle+Vehicle (saline i.p.)+vehicle (CMC), p.o.); Group 2 Idazoxan (2 mg/kg, ip)+CMC (p.o.)+vehicle (saline, i.p.); Group 3: Idazoxan (2 mg/kg, ip)+CMC (p.o.)+vehicle (saline i.p.); Group 4: Idazoxan (2 mg/kg, ip)+CMC (p.o.)+Clonidine (0.3 mg/kg, i.p.); Group 5: Idazoxan (2 mg/kg, ip)+sulfisoxazole (250 mg/kg, p.o.)+Clonidine (0.3 mg/kg, i.p.).

FIG. 10 shows the effect of naloxone on clonidine and clonidine plus sulfisoxazole on analgesia (FIG. 10A) and body temperature (FIG. 10B) in mice: Group 1: Naloxone (1.0 mg/kg, i.p.)+CMC (p.o.)+vehicle (saline i.p.); Group 2: Naloxone (1.0 mg/kg, i.p.)+CMC (p.o.)+Clonidine (0.3 mg/kg, i.p.); Group 3: Naloxone (1.0 mg/kg, i.p.)+sulfisoxazole (250 mg/kg, p.o.)+Clonidine (0.3 mg/kg, i.p.).

FIG. 11 show a comparison of the effect of clonidine plus sulfisoxazole with another ET_{A} antagonist (BMS182874) on analgesia (FIG. 11A) and body temperature (FIG. 11B) in mice: Group 1: Clonidine (0.3 mg/kg, i.p.)+BMS 182874 (2.0 μg/kg, icv); Group 2: Clonidine (0.3 mg/kg, i.p.)+BMS 182874 (10.0 μg/kg, icv); Group 3: Clonidine (0.3 mg/kg, i.p.)+BMS 182874 (50.0 pg/kg, icv).

FIG. 12 shows the effect of clonidine (1 mg/kg, ip) and BMS 182874 (50 pg, icv) on morphine (8 mg/kg, sc) analgesia. Tail flick latencies (FIG. 12A) were measured at various time intervals and antinociceptive response in each rat was converted to AUC_{0→360 min} (FIG. 12B, clonidine; FIG. 12C, BMS182874) and the values are expressed as Mean±S.E.M. (V=vehicle; B=BMS182874; I=idazoxan and M=morphine).

FIG. 13 shows the effect of clonidine (1 mg/kg, ip) and BMS182874 (50 μg, icv) on oxycodone (4 mg/kg, sc) analgesia. Tail flick latencies (FIG. 13A) were measured at various time intervals and antinociceptive response in each rat was converted to AUC_{0→360 min} (FIG. 13B, clonidine; FIG. 13C, BMS182874) and the values are expressed as Mean±S.E.M. (V=vehicle; B=BMS182874; I=idazoxan and O=oxycodone).

FIG. 14 shows the effect of yohimbine (2 mg/kg, ip) on clonidine (1 mg/kg, ip) induced potentiation of morphine (8 mg/kg, sc) analgesia or oxycodone (4 mg/kg, sc) analgesia. Tail flick latencies (FIG. 14A) were measured at various time intervals and antinociceptive response in each rat was converted to AUC_{0→360 min} (FIG. 14B, morphine; FIG. 13C, oxycodone) and the values are expressed as Mean±S.E.M. (V=vehicle; C=clonidine; Y=yohimbine; O=oxycodone and M=morphine).

FIG. 15 shows the effect of yohimbine (2 mg/kg, ip) on BMS182874 (50 μg, icv) induced potentiation of morphine (8 mg/kg, sc) analgesia or oxycodone (4 mg/kg, sc) analgesia. Tail flick latencies (FIG. 15A) were measured at various time intervals and antinociceptive response in each rat was converted to AUC_{0→360 min} (FIG. 15B, morphine; FIG. 15C, oxyocdone) and the values are expressed as Mean±S.E.M. (V=vehicle; B=BMS182874; Y=yohimbine; O=oxycodone and M=morphine).

FIG. 16 shows the effect of clonidine [0 (C0), 0.1 (C1), 0.3 (C2) and 1.0 (C3) mg/kg, ip] on morphine (8 mg/kg, sc) analgesia or oxycodone (4 mg/kg, sc) analgesia in presence of BMS182874 (50 μg, icv). Tail flick latencies (FIG. 16A, morphone; FIG. 16C, oxycodone) were measured at various time intervals and antinociceptive response in each rat was converted to AUC_{0→360 min} (FIG. 16B, morphine; FIG. 16D, oxycodone) and the values are expressed as Mean±S.E.M. (C=clonidine; B=BMS182874; O=oxycodone and M=morphine).

FIG. 17 shows the effect of clonidine (1 mg/kg, ip) and BMS182874 (50 μg, icv) alone and combined clonidine plus BMS182874 on morphine (8 mg/kg, sc) analgesia or oxycodone (4 mg/kg, sc) analgesia. Tail flick latencies (FIG. 17A) were measured at various time intervals and antinociceptive response in each rat was converted to AUC_{0→360 min} (FIG. 17B, morphine; FIG. 17C, oxyocdone)and the values are expressed as Mean±S.E.M. (V=vehicle; C=clonidine; B=BMS182874; O=oxycodone and M=morphine).

FIG. 18 shows the effect of BMS182874 (50 μg, icv) and clonidine (1 mg/kg, ip) alone and combined BMS182874 (50 μg, icv) plus clonidine (1 mg/kg, ip) on analgesia. Tail flick latencies (FIG. 18A) were measured at various time intervals and antinociceptive response in each rat was converted to AUC_{0→360 min} (FIG. 18B) and the values are expressed as Mean±S.E.M. (V=vehicle; C=clonidine; B=BMS182874).

### DETAILED DESCRIPTION OF THE INVENTION

The present invention relates to methods of treating pain using a combination of an α₂ adrenergic agonist and endothelin receptor antagonist, which produces significant analgesia and relief from pain stimulation. More specifically, the invention relates to the discovery that alpha-2 adrenergic agonists in combination with ET_{A} antagonists can act synergistically to reduce tolerance to opioid pain relievers and reduce pain symptoms.

Methods of the invention utilize a combination of an α₂ adrenergic agonist (e.g., clonidine) and an ET_{A} receptor antagonist (e.g., sulfisoxazole) that produce potent analgesia. In one aspect, methods are provided wherein sulfisoxazole and clonidine augment analgesia as demonstrated by tail flick latency in mice. The augmentation in the method is so marked that the analgesia induced by combined use of clonidine and sulfisoxazole was comparable to a high dose of morphine.

The term “treatment” as used herein, refers to preventing, reducing or otherwise ameliorating pain, or eliminating pain. As such, the term “treatment” includes both medical therapeutic and/or prophylactic administration, as appropriate. Treatment and relief of pain symptoms may be measured using pain assessment scales known in the art [see e.g., 3,5,9]. Exemplary protocols include measurement of the subjective pain threshold (visual analog scale) and the objective nociceptive flexion reflex (R III) threshold.

The term “pain” as used herein, refers to all types of pain. In one aspect, the term refers to acute and chronic pains. Exemplary types of pain include, but are not limited to, causalgia, tactile allodynia, neuropathic pain, hyperalgesia, hyperpathia, inflammatory pain, post-operative pain, chronic lower back pain, cluster headaches, postherpetic neuralgia, phantom limb and stump pain, central pain, dental pain, neuropathic pain, opioid-resistant pain, visceral pain, surgical pain, bone injury pain, diabetic neuropathy pain, post-surgery or traumatic neuropathy pain, peripheral neuropathy pain, entrapment neuropathy pain, neuropathy caused by alcohol abuse, pain from HIV infection, multiple sclerosis hypothyroidism or anticancer chemotherapy pain, pain during labor and delivery, pain resulting from burns, including sunburn, post partum pain, migraine, angina pain, and genitourinary tract-related pain including cystitis.

The term “analgesic” as used herein refer to an active agent that relieves pain in a subject. The term “opiate analgesic” or “opioid analgesic” refers to a narcotic analgesic used, for example, as an adjunct to anesthesia, or to alleviate pain. The term “non-opiate analgesic” refers to a non-narcotic agent indicated for pain.

A “therapeutically effective dose” refers to that amount of the active agent or agents that results in achieving the desired effect. Toxicity and therapeutic efficacy of such active agents are determined by standard pharmaceutical procedures in cell cultures or experimental animals, e.g., determining the LD50 (the dose lethal to 50% of the population) and the ED50 (the dose therapeutically effective in 50% of the population). The dose ratio between toxic and therapeutic effects is the therapeutic index, which is expressed as the ratio between LD50 and ED50. A high therapeutic index is preferred. The data obtained from such data is used in formulating a range of dosage for use in humans. The dosage of the active agents, in one aspect, lies within a range of circulating concentrations that include the ED50 with little or no toxicity. The dosage varies within this range depending upon the dosage form employed, and the route of administration utilized.

A “synergistic combination” of an α₂ adrenergic agonist and an endothelin receptor antagonist is a combination that has an effect that is greater than the sum of the effects of the active ingredients when administered alone.

The term “potentiate” or “potentiation” as used herein refers to the ability of an alpha-2 (α₂) adrenergic agonist or an endothelin antagonist to increase the effect of or act synergistically with an analgesic, e.g., to strengthen a biochemical or physiological effect. In one embodiment, the potentiation effectively lowers the dose of analgesic required to provide a desired pain-reducing effect. It is contemplated that potentiation occurs without affecting the cataleptic properties of the analgesic.

“Concurrent administration,” “administered in combination,” “simultaneous administration” or similar phrases mean that a composition comprising two or more agents are administered concurrently to the subject being treated. By “concurrently,” it is meant that each agent is administered at the same time or sequentially in any order at different points in time. However, if not administered at the same time, they are, in one aspect, administered sufficiently closely in time so as to provide the desired potentiation of treatment effect. Suitable dosing intervals and dosing order with such compounds will be readily apparent to those skilled in the art. It is also contemplated that two or more agents are administered in separate compositions, and in one aspect, one composition is administered prior to or subsequent to administration of the first agent. Prior administration refers to administration of the agents within the range of one day (24 hours) prior to treatment up to 30 minutes before treatment. It is further contemplated that one agent is administered subsequent to administration of the other agent. Subsequent administration is meant to describe administration from 30 minutes after administration of the first agent up to one day (24 hours) after administration of the first agent. Within 30 minutes to 24 hours may includes administration at 30 minutes, 1, 2, 3, 4, 5, 6, 7, 8, 9, 10, 11, 12, 16, 20 or 24 hours.

The term “low dose” as used herein refers to a dose of an active ingredient in a composition, wherein the amount of active ingredient in the composition is lower than that typically given in treatment of a subject. For example, the low dose of active agent may be administered in combination with a second active agent such that the active agents exhibit a synergistic effect, and the dose of each active agent in the combination treatment is lower than the dose necessary when the agent is administered not in combination with a second active ingredient. In one embodiment, the low dose of clonidine is in the range from 10 μg to about 300 μg. In a related embodiment, the low dose of sulfisoxazole is in the range from 0.1 g to about 3.0 g.

### Alpha-2 (α₂) Adrenergic Agonists

Alpha-2 (α₂) adrenergic receptors (adrenoceptors) are ubiquitously distributed in both the nervous system as well as in every other system in the body. The α₂ adrenoceptors comprise three different receptor subtypes (termed A, B, and C), are activated by the non-selective endogenous adrenergic agonists adrenaline and noradrenaline, which also activate six other adrenoceptor subtypes (U.S. Pat. No. 6,562,855). Clinical studies have shown that α₂ agonists exert powerful analgesic effects. Systemically and neuraxially administered α₂ agonists, such as clonidine and dexmedetomidine, alleviate pain in humans and in animal models. The α₂ agonists produce analgesia by a supraspinal as well as by a local spinal action (Guo et al, Anesthesiology 1991; 75: 252-6, U.S. Pat. No. 6,562,855, the disclosures of which are incorporated by reference herein in their entireties). Additional description of α₂ adrenergic agonists is found in U.S. Pat. Nos. 6,562,855, 5,605,911, and 5,980,927, the disclosures of which are incorporated by reference herein in their entireties.

Clonidine, an α₂ adrenergic agonist, has been demonstrated to produce significant analgesia in mice and rat [31]. It was also found that repeated administration of clonidine (twice daily for 7 days) produced tolerance. On the other hand, acute administration of clonidine enhanced the analgesic effect of morphine [30]. In another study, it was found that clonidine potentiated morphine analgesia but there was no cross tolerance between clonidine and morphine analgesia [38]. Measurement of pain sensitivity by the formalin test indicated that clonidine produces analgesia, and its effect was inhibited by naloxone (2 mg/kg i.p.) it is proposed that a naloxone-sensitive component of the clonidine effect is due to release of a beta-endorphin-like opioid [22]. Clonidine has also been used to suppress opiate withdrawal. These two properties make clonidine an attractive analgesic substitute in patients tolerant to opioids [27]. Clonidine has been demonstrated to enhance not only morphine analgesia, but also analgesic actions of pentazocine [13], fentanyl [9], and bupivacaine [19]. Additionally, clonidine analgesia has been found to be enhanced by amitriptyline [1].

The analgesic effect of clonidine described in animal studies was investigated in healthy volunteers in a cross-over, double-blind, placebo-controlled design where subjects received oral placebo or clonidine (0.2 mg p.o.) or clonidine and naloxone (2.8 mg i.v. in 5 h). Analgesia was assessed by measurement of the subjective pain threshold (visual analog scale) and the objective nociceptive flexion reflex (R III) threshold after transcutaneous electrical stimulations. A correlation was observed between subjective and objective thresholds (r: 0.78). Oral clonidine alone or with naloxone increased subjective and objective pain thresholds for at least 4 hours (p less than 0.01, ANOVA). Naloxone tended to reinforce clonidine analgesia. Only moderate and well tolerated side-effects were observed [32].

Studies in adults have clearly shown that addition of clonidine in preoperative, perioperative or postoperative situation leads to greater analgesic effect and lowers the incidence of side effects. Studies have also been performed in pediatric patients to determine the effect of clonidine. Extradural clonidine in randomized 45 pediatric patients aged 1-7 years was studied and it was found that the duration of postoperative analgesia with caudal bupivacaine was significantly increased by the addition of 1 μg/kg of clonidine[20]. In an 11 year old boy with second and third degree burn to 78% of body large doses of morphine produced severe side effects, which were significantly reduced by addition of low-dose intravenous clonidine [24]. However, in pediatric patients studies have also shown that midazolam is superior to oral clonidine for pre-operative sedation but induced analgesia may well be useful in pediatric anesthesia [29].

A diverse group of drugs are being used as adjuvant analgesics, although they were originally developed for a primary indication other then pain. These drugs are used to enhance analgesia under specific circumstances and some of then are used as primary analgesics [21]. Tricyclic antidepressants such as anitryptyline, nortryptyline and desipramine are effective for most neuropathic pain [36]. Bupropion, venlafaxine and duloxetine have also been found to effective in neuropathic pain management [35,37]. Antiepileptic drugs are becoming the most promising drugs for the management of neuropathic pain, gabapentin and pregabalin have both established efficacy for neuropathic pain [2,12]. Clonidine produces synergistic antinociceptive effect with opioids, in addition to being a primary analgesic [14]. Tizanidine a relatively short acting α₂ adrenergic agonist with much lower hypotensive effect than clonidine has some usefulness in pain disorders [11]. NMDA antagonists dextromethorphan, methadone, memantine, amantidine, and ketamine seem to be effective in hyperalgesic neuropathic states [3].

Clonidine, a nonopiate with antinociceptive properties, might be an alternative for postoperative analgesia free of opioid-induced side effects. Studies were conducted to investigate the analgesic properties of intravenous clonidine during the postoperative period, 50 patients, immediately after spinal fusion, were randomly assigned to two groups, blindly administered either clonidine (5 micrograms/kg infused the 1st h and then 0.3 microgram-1.kg-1.h-1 during 11 h) or a placebo. A visual analog scale graded from 0 (no pain) to 100 mm was used to assess pain before clonidine or placebo administration (T0), at the end of the loading dose (T1) and then every 2 h (T3, T5, T7, T9, and T11). Morphine (0.1 mg/kg) was administered intramuscularly after each pain measurement if the score was greater than 50 mm. No morphine was given at T0. Hemodynamics, blood gases and plasma clonidine concentrations were measured each time the pain score was measured. The pain score decreased from 42±5 to 26±3 mm (mean±standard error) in the clonidine group whereas it was unchanged in the placebo group despite a greater morphine requirement (dose for each patient: 3.8±1 vs. 10.8±1.2 mg). Clonidine delayed the onset of pain and the first request for morphine injection. Mean arterial pressure decreased to 74±2 mmHg in the clonidine group (−26±2 vs. −15±2% in the placebo group at T11) despite a significant increase in the cumulative fluid volume [3].

Studies were conducted to determine the effect of clonidine after epidural administration in 13 patients undergoing abdominal hysterectomy. A significant decrease in blood pressure and verbal analogue pain scores were observed. The degree of analgesia was moderate and short-lived following epidural clonidine (150 μg) while absorption of clonidine was very rapid from epidural space into the blood [66]. In an experimental study guanfacine was found to produce a longer duration of antinociception (guanfacine=8 h vs clonidine=5.5 h), while there were no marked hemodynamic differences between the two drugs. It is possible that because of a longer duration of action and less respiratory depression, epidural guanfacine may be superior for postoperative analgesia and chronic pain syndromes [67].

In a double-blind, controlled study, patients, undergoing total hip replacement the effectiveness of extradural clonidine infusions for postoperative analgesia and the effect of clonidine on extradural morphine was investigated. Patients were allocated randomly to receive one of two doses of extradural clonidine (25 micrograms h-1 or 50 micrograms h-1), low dose extradural morphine or a combination of morphine and clonidine. Pain scores in the morphine group were significantly greater than in the clonidine groups (P less than 0.01) and the combination group (P less than 0.05) during the first 1 h after surgery. The requirements for systemic analgesia were least in the combination and larger dose clonidine group, and the duration of effect of the initial bolus dose was significantly longer compared with the morphine and low dose clonidine groups (P less than 0.05). Arterial pressure was reduced in the clonidine groups, although the incidence of clinical hypotension was low. There were no significant differences between the groups in emetic symptoms or urinary retention [70].

It has been found that addition of oral clonidine (300 μg), 1 hour before and 12 hours after surgery reduced only slightly the requirement of morphine, however, clonidine significantly decreased heart rate and increased sedation in patients with major abdominal surgery [69].

Addition of clonidine to epidural morphine was investigated in a randomized, double blind, dose-response study in patients undergoing cesarean delivery. It was found that a low dose of 75 μg of clonidine doubled the duration of analgesia produced by 2 mg of morphine and morphine requirements during postoperative period was greatly reduced by the addition of clonidine [68].

Effect of perioperative oral clonidine on postoperative analgesia and PCA morphine requirements in adult patients after major orthopedic knee surgery was evaluated. Clonidine reduced the incidence of postoperative nausea and vomiting compared to placebo and significantly decreased PCA morphine requirements [65]. Oral clonidine (5 μg kg) premedication in 26 patients aged 37 to 60 years undergoing abdominal total hysterectomy under spinal intrathecal morphine anesthesia enhanced the postoperative analgesic effect of morphine without increasing the intensity of morphine side effects [63]. Oral clonidine (4 μg/kg) was found to reduce the PCA morphine requirement after cesarean section without compromising the condition of the fetus or newborn [64].

Patients undergoing lower abdominal surgery were recruited into a randomized double blind study. At the end of surgery Group C received an infusion of clonidine 4 micrograms kg-1 over 20 min, PCA clonidine 20 micrograms and morphine 1 mg bolus. Group M received an infusion of saline and then PCA morphine 1 mg bolus. Pain, sedation and nausea and vomiting were assessed after 12, 24 and 36 h, and satisfaction with analgesia was assessed at 36 h. Pain scores were significantly lower in Group C between 0 and 12 h, but thereafter there was no difference. Morphine consumption was the same for both groups until 24-36 h. Nausea and vomiting was significantly reduced in Group C between 0 and 24 h. Patients in group with clonidine were significantly happier with their pain relief [62].

The efficacy and side effects of a low dose of epidural morphine combined with clonidine for postoperative pain relief after lumbar disc surgery was investigated. It was found that epidural administration of morphine-clonidine significantly improved postoperative pain relief and reduced piritramide consumption as compared to epidural bupivacaine-clonidine [61].

In a double-blind randomized study, 45 patients having coronary artery bypass graft surgery were allocated randomly to receive i.v. patient-controlled analgesia (PCA) morphine (bolus, 1 mg; lock-out interval, 7 min) (control group), either alone or combined with intrathecal morphine 4 microg kg(−1) or with both intrathecal morphine 4 microg kg(−1) and clonidine 1 microg kg(−1). Intrathecal injections were performed before the induction of general anaesthesia. Pain was measured after surgery using a visual analogue scale (VAS). Morphine dosage [median (25th-75th percentiles)] was less in the first 24 h in the patients who were given intrathecal morphine+clonidine [7 (0-37) mg] than in other patients [40.5 (15-61.5) mg in the intrathecal morphine group and 37 (30.5-51) mg in the i.v. morphine group]. VAS scores were lower after intrathecal morphine+clonidine compared with the control group. Time to extubation was less after intrathecal morphine+clonidine compared with the i.v. morphine group [225 (195-330) vs 330 (300-360) min, P<0.05]. Intrathecal morphine and clonidine provide effective analgesia after coronary artery bypass graft surgery and allow earlier extubation [60].

Clonidine (120 μg) and fentanyl (50 μg) combination provided comparable extradural analgesic efficacy as compared to 0.25% bupivacaine for first stage of labor, and unwanted neurological side effects were also less [59]. However, an epidural clonidine plus morphine combination resulted in inferior analgesia and more side effects, compared with a bupivacaine plus sufentanil patient controlled regimen [58]. In another study addition of clonidine to epidural butorphanol did not enhance its analgesic effects nor did it reduced adverse effects in patients undergoing abdominal surgeries [57]. Oral clonidine premedication in healthy patients provided useful sedation and anxiolysis and stable hemodynamics, without prolongation of sensory and motor block. Side effects were observed only with clonidine 5 μg/kg dose and a 2.5 μg/kg dose of clonidine produced minimal side effects [56]. Addition of clonidine (0.75 μg/kg) proved to be better than fenanyl (0.5 μg/kg) because of lower clinically significant side effects [55]. Oral clonidine (5μg/kg) premedication reduced the induction of dose of propofol but delayed the emergence from propofol anesthesia [54].

Intraarticular administration of clonidine (150 μg) provided longer lasting pain relief postoperatively following arthroscopic knee surgery compared to 5 mg of morphine [53].

Additional α₂ adrenergic agonists, with or without imidazoline activity, contemplated for use in the invention include, but are not limited to, dexmedetomidine, detomidine, ST-91, medetomidine, brimonidine, tizanidine, mivazerol, guanabenz, guanfacine, iodoclonidine, xylazine, rilmenidine, lofexidine, azepexole, alpha-methyldopa, and alpha-methylnoradrenaline or a derivative, salt or structural analogue thereof.

### Endothelin Receptor Antagonists

Endothelins (e.g., ET-1, ET-2 and ET-3), which bind to both ET_{A} and ET_{B}, are potent vasoconstrictors. Endothelins are synthesized as preprohormones and post-translationally processed to active peptides. ET-1 processing has been best characterized and begins with the 212 amino acid peptide (preproET-1), which is then proteolytically cleaved by endopeptidases to big ET-1 (proET-1). The 39 amino acid proET-1 is cleaved by the metalloendoprotease endothelin converting enzyme (ECE), resulting in the 21 amino acid protein with potent biologic functions (Fagan et al., Respiratory Research 2:90-101, 2001).

As used herein, “endothelin antagonist” and “endothelin receptor antagonist” are used interchangeably. Endothelin receptor antagonists are used to treat acute heart failure, congestive/chronic heart failure, pulmonary arterial hypertension, pulmonary edema, subarachnoid hemorrhage, chronic obstructive pulmonary disease, myocardial infarction, acute cerebral ischemia, acute coronary syndromes, acute renal failure, post-operative treatment in liver operations, and prostate cancer.

Studies indicate that two structurally different ET_{A} receptor antagonists, BQ123 (a peptide) and BMS182874 (a non-peptide) potentiated the analgesic response of morphine in both mice and rats [5-7,16,26,33]. An interaction between clonidine and ET in the cardiovascular effects involving sympathetic nervous system has also been reported [15,17,18,28]. Endothelin-1 (ET-1) has also been found to potentiate hypotension produced by clonidine [23]. However, there is no report on the effect of ET_{A} receptor antagonists on clonidine induced analgesic effect. Sulfisoxazole has been found to be the most active sulfanilamide with an IC₅₀ value of 0.60 μM and 22 μM for ET_{A} and ET_{B} receptors, respectively [8]. Therefore, the present study was performed to determine the effect of sulfisoxazole on analgesic effect when administered in combination with clonidine. The results of the studies demonstrated that a combination of clonidine and sulfisoxazole produces potent analgesia equivalent to a high dose of morphine (See Example 5).

Sulfisoxazole, a weak endothelin A antagonist, is extensively bound to plasma proteins and following an oral dose of 2 to 4 grams peak plasma concentration of 110 to 250 μg/ml are found in 2 to 4 hours. Concentration of sulfisoxazole in urine exceeds that of blood and in the cerebrospinal fluid it averages about a third of blood concentration. Most of the drug (95%) is excreted in urine by kidney in 24 hours (Goodman Gilmans 1990 8^{th} Edition).

Sulfisoxazole is largely confined to the extracelluar space and attains concentrations in the cerebrospinal fluid that may range between 10 and 80% of that in the blood (Goodman Gilmans 1990 8^{th} Edition). Free sulfisoxazole blood levels of 50 to 150 μg/ml are considered therapeutically effective for most infections, with blood levels of 120 to 150 μg/ml being optimal for serious infections. The maximum level should be 200 μg/ml in blood because adverse reactions occur more frequently above this concentration (PDR 59^{th} Edition 2005). Multiple oral dose administration of 500 mg was given four times a day to healthy volunteers, the average steady state plasma concentration of sulfisoxazole ranged from 49.9 to 88.8 μg/ml (mean 63.4 μg/ml) (Oie et al., J Pharmacokinetics Biopharm 1982: 10: 157-172).

An interaction between propofol and sulfisoxazole in mice has been observed. Impairment of righting reflex by propofol was significantly enhanced by pretreatment with sulfisoxazole. Sulfisoxazole by itself did not produce any effect and it did not produce any change in total plasma concentration and protein binding of propofol [51].

Sulfisoxazole has been found to protect retina from ischemic insults occurring in glaucoma, by attenuating the elevation of nitric oxide and the reduction in numbers of GABA-containing neurons caused by lipopolysaccharaide (LPS) [52]. It has also been found that topical application of clonidine protects the rat retina from ischemia/reperfusion by stimulating u₂-adrenergic receptors, this protective effect was selectively attenuated by yohimbine or rauwolscine confirming the involvement of α₂-adrenergic receptors [50].

The studies relating to clonidine and sulfisoxazole are clinically important because several ET_{A} receptor antagonists are being evaluated for use in the treatment of pulmonary arterial hypertension, congestive heart failure, stroke, reclosure of coronary arteries after balloon angioplasty, hypertension, and cancer pain management. The results herein provide evidence that sulfisoxazole, a weak ET_{A} receptor antagonist, when combined with the α₂ adrenergic agonist clonidine, produced unexpected analgesic effect. It is contemplated that studies are also carried out with additional ET_{A} receptor antagonist combined with α₂ adrenergic agonists, including but not limited to clonidine, to assess whether combinations of these agents produce augmentation of analgesic effect and that such combinations can be used as safe non-opiate analgesics. The results described herein provide an additional target by which nociception can be modulated and may provide a novel approach in the management of chronic pain in patients.

Examples of endothelin receptor antagonists useful in the present invention include, but are not limited to, sulfisoxazole, atrasentan, tezosentan, bosentan, sitaxsentan, enrasentan, BMS 207940 (Bristol-Myers Squibb), BMS 193884, BMS 182874, J 104132 (Banyu Pharmaceutical), VML 588/Ro 61 1790 (Vanguard Medica), T-0115 (Tanabe Seiyaku), TAK 044 (Takeda), BQ 788, TBC2576, TBC3214, PD180988, ABT 546, SB247083, RPR118031A and BQ123. BQ123 is a specific endothelin A antagonist, and is the sodium salt of cyclo(D-Trp-D-Asp-Pro-D-Val-Leu). Other useful nonlimiting endothelin antagonists have the designations YM 598, LU 135252, PD 145065, A 127722, ABT 627, A 192621, A 182086, TBC3711, BSF208075, S 0139, and SB209670. Additional useful endothelin A antagonists can be found in U.S. Patent Application Publication No. US 2002/0082285 A1 and U.S. patent application Ser. No. 10/659,579, each incorporated herein by reference.

In addition to a conventional endothelin receptor antagonist, a compound that inhibits the formation of endogenous endothelin also can be used as the endothelin receptor antagonist in the present invention. Such compounds are useful because they prevent endothelin formation and therefore decrease the activity of endothelin receptors. One class of such compounds is the endothelin converting enzyme (ECE) inhibitors.

Useful ECE inhibitors include, but are not limited to, [DVal22, Phe33] big endothelin-1 (16-38), human (i.e., His-Leu-Asp-Ile-Ile-Trp-DVal-Asn-Thr-Pro-Glu-His-Val-Val-Pro-Tyr-Gly-Phe-Gly-Ser-Pro-Arg-Ser); [DVal22] big endothelin (16-38), human (i.e., His-Leu-Asp-Ile-Ile-Trp-DVal-Asn-Thr-Pro-Glu-His-Val-Val-Pro-Try-Gly-Leu-Gly-Ser-Pro-Arg-Ser); [Phe22] big endothelin-l (19-37), human (i.e., Ile-Ile-Trp-Phe-Asn-Thr-Pro-Glu-His-Val-Val-Pro-Tyr-Gly-Leu-Gly-Ser-Pro-Arg); and phosphoramidon (i.e., N-(a-rhamnopyranosyloxyhydroxyphosphinyl)-Leu-Trp).

### Opiate Analgesics

Available opiate and opioid analgesics are derivatives of five chemical groups (i.e., phenanthrenes, phenylheptylamines, phenylpiperidines, morphinans, and benzomorphans). Pharmacologically, opiates and nonopiates differ significantly in activity. Some are strong agonists (morphine), others are moderates-to-mild agonists (codeine). In contrast, some opiate derivatives exhibit mixed agonist-antagonist activity (nalbuphine), whereas others are opiate antagonists (naloxone). Morphine is the prototype of the opiate and opioid analgesics, all of which have similar actions on the central nervous system.

Morphine is chemically derived from opium. Other drugs, such as heroin, are processed from morphine or codeine. Such opiates have been used both medically and nonmedically for centuries. By the early 19th century, morphine had been extracted in a pure form suitable for solution. With the introduction of the hypodermic needle, injection of a morphine solution became the common method of administration. Of the twenty alkaloids contained in opium, only codeine and morphine are still in widespread clinical use.

The opium group of narcotic drugs are among the most powerfully acting and clinically useful drugs producing depression of the central nervous system. Drugs of this group are used principally as analgesics, but possess numerous other useful properties. Morphine, for example, is used to relieve pain, induce sleep in the presence of pain, check diarrhea, suppress cough, ease dyspnea, and facilitate anesthesia.

When morphine and related compounds are administered over a long period of time, tolerance to the analgesic effect develops, and the dose then must be increased periodically to obtain equivalent pain relief. Eventually, tolerance and physical dependence develop, which, combined with euphoria, result in excessive use and addiction of those patients having susceptible personalities. For these reasons, morphine and its derivatives must be used only as directed by a physician (i.e., not in greater dose, more often, or longer than prescribed), and should not be used to treat pain when a different analgesic will suffice.

It is contemplated that one or more alpha-2 (α₂) adrenergic agonist and/or one more endothelin antagonist are useful to potentiate the analgesic effects of an opiate analgesic. It is further contemplated that the combination of one or more alpha-2 (α₂) adrenergic agonist and one or more endothelin antagonist is useful to potentiate the analgesic effect of an opiate analgesic.

Opiate analgesics include, but are not limited to, (a) opium; (b) opium alkaloids, such as morphine, morphine sulfate, codeine, codeine phosphate, codeine sulfate, diacetylmorphine, morphine hydrochloride, morphine tartrate, and diacetylmorphine hydrochloride; and (c) semisynthetic opiate analgesics, such as dextromethorphan hydrobromide, hydrocodone bitartrate, hydromorphone, hydromorphone hydrochloride, levorphanol tartrate, oxymorphone hydrochloride, and oxycodone hydrochloride.

### Formulation of Pharmaceutical Compounds

The active agents of the present invention, i.e., an α₂ adrenergic agonist and an endothelin receptor antagonist described herein, can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active agents into preparations which can be used pharmaceutically.

These pharmaceutical compositions can be manufactured in a conventional manner, e.g., by conventional mixing, dissolving, granulating, dragee-making, emulsifying, encapsulating, entrapping, or lyophilizing processes. Proper formulation is dependent upon the route of administration chosen.

Pharmaceutical compositions comprising the active agents described herein are contemplated, and in one aspect the compounds are formulated with pharmaceutically acceptable diluents, adjuvants, excipients, and/or carriers. The phrase “pharmaceutically or pharmacologically acceptable” refers to molecular entities and compositions that do not produce adverse, allergic, or other untoward reactions when administered to an animal or a human, e.g., orally, topically, transdermally, parenterally, by inhalation spray, vaginally, rectally, or by intracranial injection. The term parenteral as used herein includes subcutaneous injections, intravenous, intramuscular, intracistemal injection, or infusion techniques. Administration by intravenous, intradermal, intramusclar, intramammary, intraperitoneal, intraarticular, intrathecal, retrobulbar, intrapulmonary injection and or surgical implantation at a particular site is contemplated as well. Generally, the compositions prepared are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art.

The pharmaceutical compositions described above for use in the methods may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any known method, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for controlled release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. When administered in tablet form, the composition can additionally contain a solid carrier, such as a gelatin or an adjuvant. The tablet, capsule, and powder contain about 5% to about 95% of an active agent of the present invention, and preferably from about 25% to about 90% compound of the present invention. When administered in liquid form, a liquid carrier, such as water, petroleum, or oils of animal or plant origin, can be added. The liquid form of the composition can further contain physiological saline solution, dextrose or other saccharide solutions, or glycols. When administered in liquid form, the composition contains about 0.5% to about 90% by weight of active agents, and preferably about 1% to about 50% of an active agents.

Aqueous suspensions may contain the active agents in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions useful in the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and/or coloring agents. The compositions may also be in the form of suppositories for rectal administration. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols, for example.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must also be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Administration and Dosing

The pharmaceutical compositions include those wherein the active ingredients are administered in a therapeutically effective amount to achieve their intended purpose. Determination of a therapeutically effective amount is well within the capability of those skilled in the art, especially in light of the disclosure provided herein, and giving consideration to the effect desired to be achieved, the route of administration, and the condition of the recipient patient.

The exact formulation, route of administration, and dosage is determined by an individual physician in view of the patient's condition. Dosage amount and interval can be adjusted individually to provide levels of the active agents that are sufficient to maintain therapeutic or prophylactic effects.

It is contemplated that the subject treated using the methods described herein is a mammalian subject. The mammalian subject may be human, or any non-human animal model for human medical research, or an animal of importance as livestock or pets, for example, companion animals.

Administration of the pharmaceutical composition(s) can be performed before, during, or after the onset of pain.

The active agents can be administered by any suitable route, for example by oral, buccal, inhalation, sublingual, rectal, vaginal, intracisternal through lumbar puncture, transurethral, nasal, percutaneous, i.e., transdermal, or parenteral (including intravenous, intramuscular, subcutaneous, and intracoronary) administration. Parenteral administration can be accomplished using a needle and syringe, or using a high pressure technique, like POWDERJECT™.

The active agents of the present invention can be administered alone, or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers comprising excipients and auxiliaries that facilitate processing of the active agents into preparations which can be used pharmaceutically. Pharmaceutical compositions can be manufactured in a conventional manner, as described herein and known in the art.

The amount of pharmaceutical composition administered is dependent on the subject being treated, on the subject's weight, the severity of the affliction, the manner of administration, and the judgment of the prescribing physician.

For veterinary use, the active ingredients are administered as a suitably acceptable formulation in accordance with normal veterinary practice. The veterinarian can readily determine the dosing regimen and route of administration that is most appropriate for a particular animal.

In one embodiment, for administration to a human in the curative or prophylactic treatment of pain, oral dosages of α₂ adrenergic agonists and endothelin receptor antagonist, individually generally are about 10 to about 200 mg daily for an average adult patient (70 kg), typically divided into two to three doses per day. When the active agents are administered in conjunction with an opiate analgesic, for a typical adult patient, individual tablets or capsules contain about 0.1 to about 200 mg opioid analgesic, about 5 μg/kg or 300 μg α₂ adrenergic agonist, and/or about 0.1 to about 50 mg endothelin antagonist, in a suitable pharmaceutically acceptable vehicle or carrier, for administration in single or multiple doses, once or several times per day. Dosages for intravenous, buccal, or sublingual administration typically are about 0.1 to about 10 mg/kg per single dose as required. In practice, the physician determines the actual dosing regimen which is most suitable for an individual patient, and the dosage varies with the age, weight, and response of the particular patient. The above dosages are exemplary of the average case, but there can be individual instances in which higher or lower dosages are merited, and such are within the scope of this invention.

In one embodiment, the preparation containing clonidine, sulfisoxazole or clonidine and sulfisoxazole is prepared in the form of a tablet or capsule.

Clonidine has been used orally in the doses of 5 μg/kg or 300 μg total dose as an adjuvant for analgesia. Overall clinical studies using clonidine have found that 1-5 μg/kg dose of clonidine is effective in producing analgesia. Experiments disclosed herein in rat showed that a combination of 2 mg/kg of clonidine and 1000 mg/kg of sulfisoxazole was effective in producing significant analgesia. Sulfisoxazole has been used in does of 2 to 4 grams orally.

In one embodiment, the α₂ adrenergic agonist is administered at a dose of about 50 μg, about 110 μg, about 150 μg, about 200 μg, about 250 μg, about 300 μg, about 350 μg, about 400 μg, about 450 μg, about 500 μg, about 750 μg or about 1000 μg. In a related embodiment, clonidine is administered within a dose range of about 10 to about 500 μg, about 10 μg to about 300 μg about 75 to about 300 μg, or about 100 to about 250 μg.

In a further embodiment, the endothelin receptor antagonist is administered at a dose of about 0. 1, about 0.5, about 1, about 2, about 3, about 4, about 5, about 10, about 15, about 20, about 25, about 30, about 35, about 40, about 45 or about 50 mg, or within a dose range from about 0.1 mg to about 50 mg. In a related embodiment, sulfisoxazole is administered within a dose range from 2 to 4 grams, from 750 mg to 1.5 g, from 1 to 2 grams, from 0.1 to 3 grams.

It is contemplated that a ratio of α₂ adrenergic agonist to endothelin antagonist administered to a subject effective to treat pain ranges from 1:500 to 1:50,000. In one embodiment, a ratio of clonidine to sulfisoxazole ranging from 1:500 to 1:50,000 should be effective in producing analgesia. It is contemplated that if a more potent endothelin receptor antagonist is used then the ratio of clonidine to endothelin antagonist will change and may be in the range of 1:100 to 1:1000. The invention provides for administration of the composition(s) wherein the ratio of alpha-2 adrenergic agonist to endothelin antagonist is in the range of 1:500 to 1:50,000, 1:500 to 1:20,000, 1:500 to 1:10,000, 1:500 to 1:5,000, 1:500 to 1:2,500, 1:100 to 1:1000, and 1:100 to 1:500.

It is contemplated that the α₂ adrenergic agonist and the endothelin receptor antagonist are administered either concurrently or separately. Further, the α₂ adrenergic agonist and endothelin receptor antagonist may be administered in a single composition or in separate compositions. In concurrent administration, a composition comprising one or more α₂ adrenergic agonist and/or one or more endothelin receptor antagonist are administered such that each agent is administered at the same time or sequentially in any order at different points in time. However, if not administered at the same time, they are, in one embodiment, administered sufficiently closely in time so as to provide the desired potentiation of treatment effect. It is also contemplated that when one or more α₂ adrenergic agonist and/or one or more endothelin receptor antagonist are administered in separate compositions, and in one aspect, one composition is administered prior to or subsequent to administration of the first agent. Prior administration refers to administration of the agents within the range of one day (24 hours) prior to treatment up to 30 minutes before treatment. It is further contemplated that one agent is administered subsequent to administration of the other agent. Subsequent administration is meant to describe administration from 30 minutes after administration of the first agent up to one day (24 hours) after administration of the first agent. Within 30 minutes to 24 hours may includes administration at 30 minutes, 1, 2, 3, 4, 5, 6, 7, 8, 9, 10, 11, 12, 16, 20 or 24 hours.

Administration of the compositions may be as necessitated by the subjects pain symptoms. In one embodiment, the agents are administered 4× daily, 2× daily, daily, every 48 hours, every 3 days, every 4 days, every 7 days, or every 14 days. Moreover, the administration may take place at multiple sites if necessary, and may be administered systemically or locally to the site of pain.

It is contemplated, in one embodiment, that the endothelin receptor antagonist is administered prior to the α₂ adrenergic agonist. In a related embodiment, the α₂ adrenergic agonist is administered prior to the endothelin receptor antagonist. It is further contemplated that when one or more α₂ adrenergic agonist and/or one or more endothelin antagonist are administered in conjunction with an opiate analgesic, the opiate analgesic is administered prior to the α₂ adrenergic agonist and endothelin receptor antagonist, is administered subsequent to the α₂ adrenergic agonist and endothelin receptor antagonist, or may be administered subsequent to administration with one agent and prior to administration with the other agent, wherein the α₂ adrenergic agonist or endothelin receptor antagonist may be administered prior to or subsequent to the opiate analgesic.

Suitable dosing intervals and dosing order with such compounds will be readily apparent to those skilled in the art.

### Methods of Treating Pain

The present invention provides methods for alleviating and treating symptoms that arise in a subject experiencing pain. In one aspect, the invention provides a method of treating or preventing pain comprising administering to a mammal a therapeutically effective amount of an α₂ adrenergic agonist and a therapeutically effective amount of an endothelin receptor antagonist. In a related embodiment, the treatment of pain may further comprise administration of an opiate analgesic in addition to the α₂ adrenergic agonist and endothelin receptor antagonist.

The causes of pain include, but are not limited to inflammation, injury, disease, muscle spasm and the onset of a neuropathic event or syndrome. Acute pain is usually self-limited, whereas chronic pain generally persists for 3 months or longer and can lead to significant changes in a patient's personality, lifestyle, functional ability and overall quality of life. Ineffectively treated pain can be detrimental to the person experiencing it by limiting function, reducing mobility, complicating sleep, and interfering with general quality of life.

Inflammatory (nociceptive) pain can occur when tissue is damaged, as can result from surgery or due to an adverse physical, chemical or thermal event or to infection by a biologic agent. Neuropathic pain is a persistent or chronic pain syndrome that can result from damage to the nervous system, the peripheral nerves, the dorsal root ganglion or dorsal root, or to the central nervous system. Neuropathic pain syndromes include allodynia, various neuralgias such as post herpetic neuralgia and trigeminal neuralgia, phantom pain, and complex regional pain syndromes, such as reflex sympathetic dystrophy and causalgia. Causalgia is characterized by spontaneous burning pain combined with hyperalgesia and allodynia. Hyperalgesia is characterized by extreme sensitivity to a painful stimulus. (Meller et al., Neuropharmacol. 33:1471-8, 1994). This condition can include visceral hyperalgesia which generates the feeling of pain in internal organs. Neuropathic pain also includes hyperpathia, wherein a stimulus that is normally innocuous if given for a prolonged period of time results in severe pain.

In one embodiment, the pain to be treated is chronic pain or acute pain. In a related embodiment, the pain is selected from the group consisting of causalgia, tactile allodynia, neuropathic pain, hyperalgesia, hyperpathia, inflammatory pain, post-operative pain, chronic lower back pain, cluster headaches, postherpetic neuralgia, phantom limb and stump pain, central pain, dental pain, neuropathic pain, opioid-resistant pain, visceral pain, surgical pain, bone injury pain, diabetic neuropathy pain, post-surgery or traumatic neuropathy pain, peripheral neuropathy pain, entrapment neuropathy pain, neuropathy caused by alcohol abuse, pain from HIV infection, multiple sclerosis hypothyroidism or anticancer chemotherapy pain, pain during labor and delivery, pain resulting from burns, including sunburn, post partum pain, migraine, angina pain, and genitourinary tract-related pain including cystitis.

It is contemplated that the combination of α₂ adrenergic agonist and endothelin receptor antagonist results in a synergistic effect such that lower doses f each compound may be used in combination compared to doses of each compound when given alone. Further, it is contemplated that either administration of an α₂ adrenergic agonist or an endothelin receptor antagonist alone may potentiate the effects of an opioid analgesic, such that lower doses of the opioid are necessary to effectively treat the symptoms of pain. Further, it is contemplated that α₂ adrenergic agonist and endothelin receptor antagonist both administered in conjunction with an opiate analgesic also potentiate the effects of the opioid and reduce the amount of opioid necessary to alleviate pain.

Treatment of chronic pain in human patients is carried out generally as described in U.S. Pat. No. 6,372,226. In one aspect, a patient experiencing acute inflammatory pain, neuropathic pain, spastic conditions, or other chronic pain from an injury is treated by intrathecal administration, for example by spinal tap to the lumbar region, with an appropriate dose of a composition described herein for use in a method of the invention. In an additional example, if the subject suffers from arthritis or other joint pain, compositions are administered intraarticularly. The particular dose and site of injection, as well as the frequency of administrations, depend upon a variety of factors within the skill of the treating physician.

Amelioration of pain symptoms is measured using methods known in the art, including the visual analog scale (VAS), the verbal rating scale (VRS) and the numerical rating scale (NRS) (Williamson et al., J Clin Nurs. 14:798-804, 2005; Carlsson, A., Pain. 1983 16:87-101, 1983). For the visual analog scale, the verbal rating scale, and the numeric rating scale, generally, patients are asked to rate their pain on a numeric scale before and after pain stimulus. Chronic pain is also assessed by an objective scaled test such as the Leeds Assessment of Neuropathic Symptoms and Signs (LANSS) Pain Scale (Bennett, M. Pain. 92:147-157, 2001). A decrease in hypersensitivity to pain stimulus after treatment with a composition comprising an α₂ adrenergic agonist and/or an endothelin receptor antagonist indicates that interfering with normal activity a α₂ adrenergic receptors and/or endothelin receptors alleviates symptoms associated with chronic pain. It another aspect of the invention, the compositions described herein are administered in conjunction with another pain medications as described above, wherein the therapies provide a synergistic effect in relieving symptoms of chronic pain.

Improvement in pain is measured at varying timepoints after administration of analgesic is administered and the reduction in pain based on the measurement scale is assessed. In one embodiment, assessment of pain symptoms is carried out every 1, 2, 3, 4, 5, 6 or 8 weeks, or as determined by a treating physician. In one embodiment, the improvement in pain symptoms in a subject, when compared to assessment of pain symptoms before treatment, may be at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% as measured using art-recognized pain scales.

### Kits

As an additional aspect, the invention includes kits which comprise one or more compounds or compositions packaged in a manner which facilitates their use to practice methods of the invention. In a simplest embodiment, such a kit includes a compound or composition described herein as useful for practice of a method of the invention (e.g., a composition comprising an α₂ adrenergic agonist and a composition comprising an endothelin receptor antagonist, or a composition comprising both an α₂ adrenergic agonist and an endothelin receptor antagonist), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration. In another embodiment, the kit may also comprise one or more opioid analgesics.

Additional aspects and details of the invention will be apparent from the following examples, which are intended to be illustrative rather than limiting.

### EXAMPLES

### Example 1

### Materials and Methods

Animals: Male Swiss Webster mice weighing 25 to 30 g (Harlan, Indianapolis, Ind.) were used. The animals were housed five per cage in a room with controlled ambient temperature (23±1° C.), humidity (50±10%) and twelve-hour light/dark cycle (6.00 AM to 6.00 PM). Food and water were made available ad libitum. Experiments were carried out after the animals had been acclimated to this environment for at least 4 days. Animal care and use for experimental procedures were approved by the Institutional Animals Care and Use. All anesthetic and surgical procedures were in compliance with the guidelines established by the Animal Care Committee.

Drugs: Morphine sulfate (Mallinckrodt Chemical Co., St. Louis, Mo.) was dissolved in distilled deionized pyrogen-free water and injected subcutaneously (s.c.). Sulfisoxazole, 4-amino-N-(3,4-dimethyloxazol-5-yl)-benzenesulfonamide (Sigma Chemical Company, St. Louis, Mo.) was dissolved in carboxymethyl cellulose and administered orally. Clonidine, N-(2,6-dichlorophenyl)-4,5-dihydro-1H-imidazol-2-amine (Sigma Chemical Company, St. Louis, Mo.) was dissolved in sterile saline and injected intraperitoneally (i.p.).

Statistics: All data values are presented as mean±SEM. ANOVA followed by post-hoc test (Bonferroni's Test) were used to test differences within and between groups. A level of P<0.05 was considered significant.

### Example 2

### Determination of Tail-Flick Latency

Antinociceptive response to morphine was determined by tail-flick latency method of D'Amour and Smith[10]. Application of thermal stimulation (focused light) to the tail of an animal provoked withdrawal of the tail by a brief vigorous movement. The reaction time of this movement was recorded as tail-flick latency by using an analgesiometer. Tail-flick latencies to thermal stimulation (focused light) were determined before and at 30, 60, 90, 120, 180, and 240 min after injection of morphine or saline. A cutoff time of 10 sec was used to prevent damage to the tail. Tail flick latency values were subtracted from the basal latency and the differential values were used to calculate the area under the curve (AUC). Antinociceptive response in each mouse was converted to AUC_{0→240 min} and expressed as mean±S.E.M.

### Example 3

### Determination of Effect of Clonidine on Morphine Antinociception

To determine the effect of clonidine on morphine induced antinociception, mice were divided into the following four groups: group 1 received vehicle (saline i.p.)+vehicle (saline s.c.); group 2 received vehicle+morphine (4 mg/kg, s.c.); group 3 received clonidine (2 mg/kg, i.p.)+vehicle (saline s.c.); and group 4 received clonidine (2 mg/kg, i.p.)+morphine (4 mg/kg, s.c.). Morphine or vehicle was administered 30 min after clonidine administration.

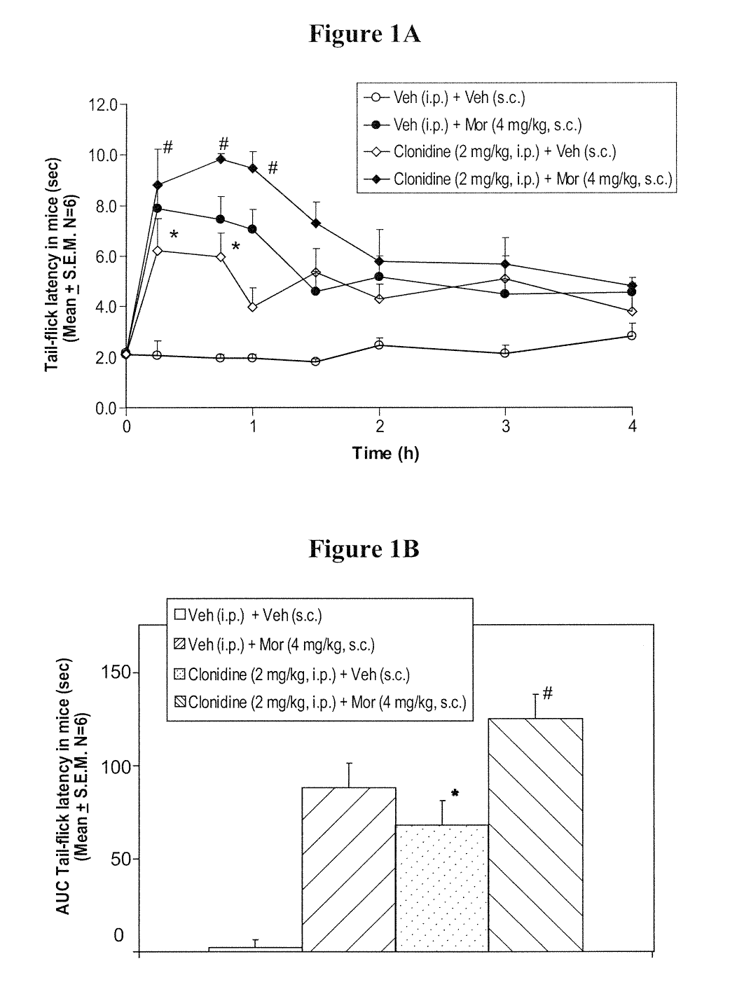

Baseline tail-flick latency without any drug treatment was 1.5 to 2.3 sec. In the control group (vehicle+vehicle), tail-flick latency did not change from baseline values over the duration of 4 hours. However, morphine (4 mg/kg, s.c.) produced a significant increase in tail flick latency. Clonidine (2 mg/kg, i.p.) produced an increase in tail flick latency and when morphine was administered in clonidine treated mice tail flick latency was further potentiated compared to morphine (FIG. 1A). The AUC observed in the morphine (4 mg/kg, s.c.) treated group was 88.4±12.4. Clonidine produced analgesia and an AUC of 68.4±12.3 was observed which was significantly higher than control. Clonidine pretreatment produced a significant potentiation of morphine analgesia and an AUC of 125.4±12.3 was observed. Clonidine significantly (42%) enhanced the antinociception produced by morphine (FIG. 1B).

### Example 4

### Determination of Effect of Sulfisoxazole on Morphine Antinociception

To determine the effect of sulfisoxazole on morphine induced antinociception, mice were divided into the following four groups: group 1 received vehicle (oral carboxymethyl cellulose (CMC))+vehicle (saline s.c.); group 2 received vehicle (CMC)+morphine (4 mg/kg, s.c.); group 3 received sulfisoxazole (500 mg/kg, oral)+vehicle (saline s.c.); and group 4 received sulfisoxazole (500 mg/kg, oral)+morphine (4 mg/kg, s.c.). Morphine or vehicle was administered 30 min after sulfisoxazole administration.

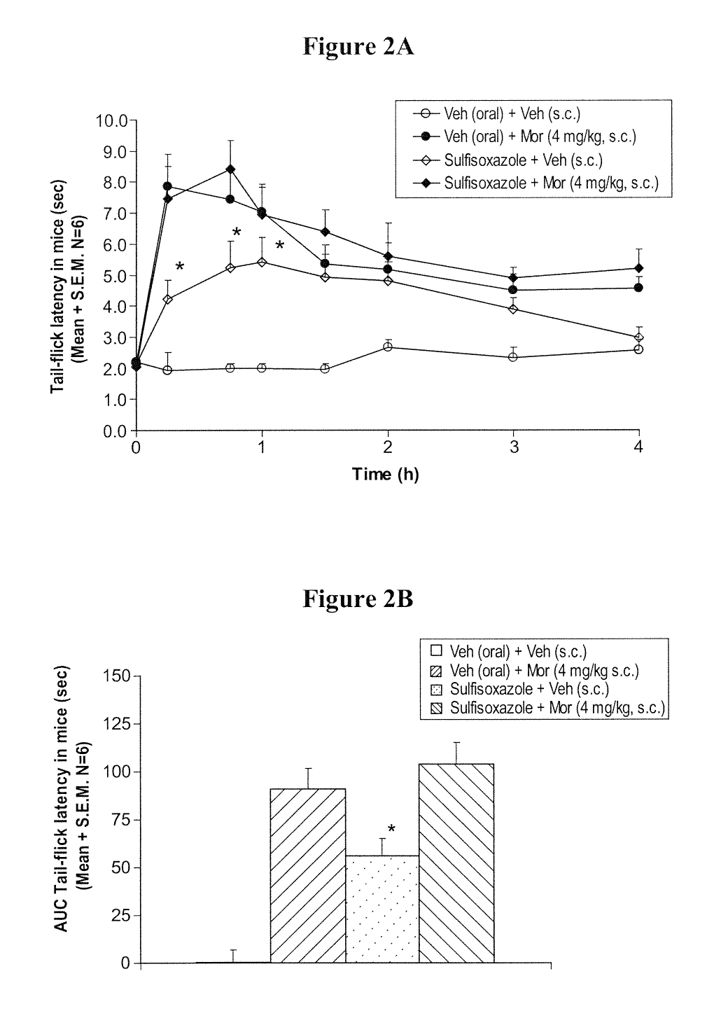

Baseline tail-flick latency without any drug treatment was 2.0 to 2.4 sec. In the control group (vehicle+vehicle), tail-flick latency did not change from baseline values over the duration of 4 hours. However, morphine (4 mg/kg, s.c.) produced a significant increase in tail flick latency. Sulfisoxazole (500 mg/kg, oral) produced an increase in tail flick latency. However, sulfisoxazole (500 mg/kg, oral) pretreatment did not produce any effect on morphine induced increase in tail flick latency in mice, indicating that sulfisoxazole when administered in the dose of 500 mg/kg, oral did not potentiate morphine analgesia (FIG. 2A). Sulfisoxazole (56.0±9.1) produced an increase in AUC compared to control (0.3±6.4) indicating that sulfisoxazole produced mild analgesic effect. Morphine (4 mg/kg, s.c.) produced significant analgesia and an AUC of 91.0±10.8 was observed. However, sulfisoxazole did not augment morphine analgesia (AUC 103.8±11.3) and only 14% potentiation of antinociception produced by morphine was observed (FIG. 2B).

### Example 5

### Determination of Effect of Clonidine Plus Sulfisoxazole on Morphine Antinociception

To determine the effect of clonidine plus sulfisoxazole on morphine induced antinociception, mice were divided into the following groups:

Study 1. Mice were divided in to three groups: group 1 received clonidine (2 mg/kg, i.p.) plus sulfisoxazole (500 mg/kg, oral)+vehicle (V) (saline s.c.); group 2 received clonidine (2 mg/kg, i.p.) plus sulfisoxazole (500 mg/kg, oral)+morphine (8 mg/kg, s.c.); group 3 received vehicle (saline, i.p.) plus vehicle (carboxymethyl cellulose, oral)+morphine (8 mg/kg, s.c.) and group 4 received vehicle (saline, i.p.) plus vehicle (carboxymethyl cellulose, oral)+vehicle (saline, s.c.);. Morphine or vehicle was administered 30 min after clonidine plus sulfisoxazole administration.

Study 2. Mice were divided in to six groups: group 1 received vehicle (saline i.p.) plus vehicle (carboxymethyl cellulose (CMC))+vehicle (saline s.c.); group 2 received clonidine (2 mg/kg, i.p.) plus vehicle (CMC)+vehicle (saline s.c.); group 3 received sulfisoxazole (1000 mg/kg, oral) plus vehicle (saline i.p.)+vehicle (saline s.c.); group 4 received vehicle (saline i.p.) plus vehicle (CMC)+morphine (8 mg/kg s.c.); group 5 received clonidine (2 mg/kg, i.p.) plus sulfisoxazole (1000 mg/kg, oral)+vehicle (saline s.c.); and group 6 received clonidine (2 mg/kg, i.p.) plus sulfisoxazole (1000 mg/kg, oral)+morphine (8 mg/kg, s.c.). Morphine or vehicle was administered 30 min after clonidine plus sulfisoxazole administration.

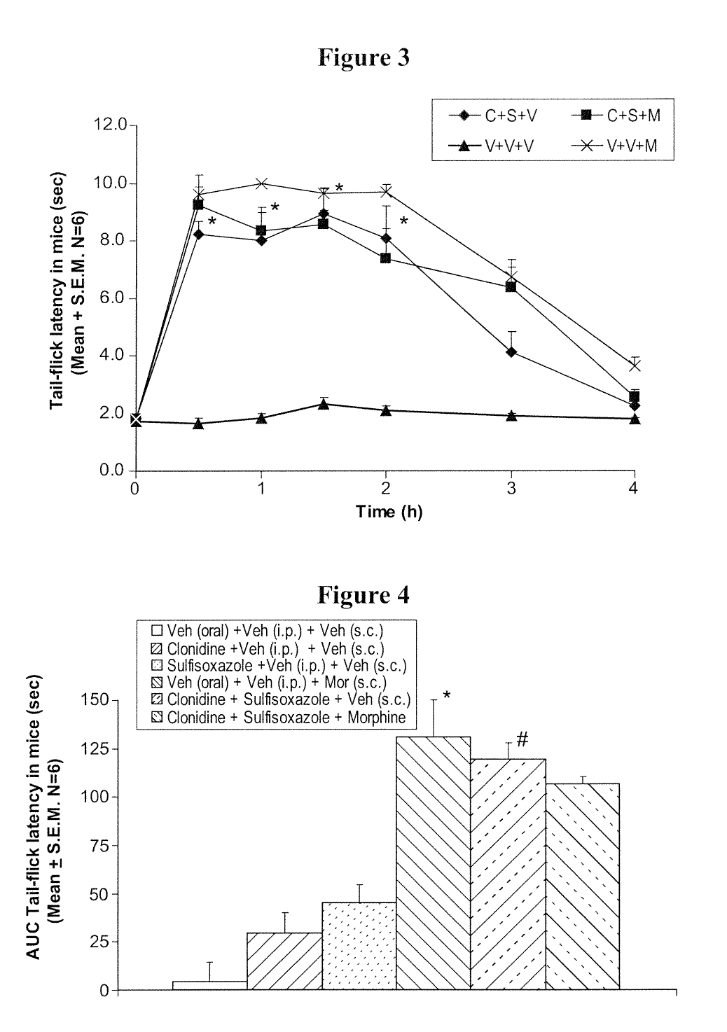

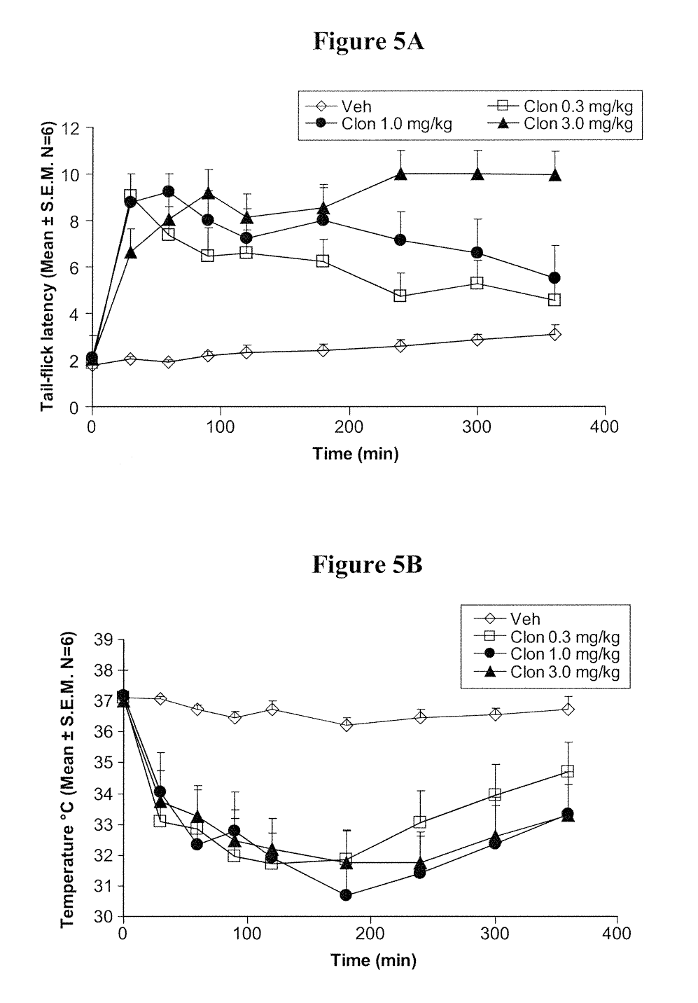

Baseline tail-flick latency without any drug treatment was 1.5 to 2.0 sec. In the control group (vehicle+vehicle), tail-flick latency did not change from baseline values over the duration of 4 hours. However, sulfisoxazole (500 mg/kg, oral) plus clonidine (2 mg/kg, i.p.) without any presence of morphine produced a significant increase in tail flick latency. Administration of sulfisoxazole (500 mg/kg, oral) plus clonidine (2 mg/kg, i.p.) did not potentiate the effect of morphine and no further increase in tail flick latency was observed (FIG. 3). It could be possible that further potentiation was not observed because the cutoff point was 10 sec and any increase beyond 10 seconds will not be observed in the present technique. The finding that two non-opiates clonidine and sulfisoxazole when administered together produced marked analgesia comparable to that of a high dose of morphine was very interesting. Further studies using a higher dose of sulfisoxazole (1000 mg/kg, oral) alone and in combination with clonidine (2 mg/kg, i.p.) were conducted. It was found that clonidine (2 mg/kg, i.p.) produced an increase in AUC of tail flick latency compared to control mice and similarly, sulfisoxazole (1000 mg/kg, oral) produced an increase in AUC of tail flick latency compared to control mice. Morphine (8 mg/kg, sc) produced significant increase in tail flick latency and AUC of 130.9±19.2 was observed. It was interesting to observe that sulfisoxazole (1000 mg/kg, oral) in combination with clonidine (2 mg/kg, i.p.) produced a significant increase in tail flick latency and the AUC observed in this group (119.1±8.9) was equal to that of a high dose of 8 mg/kg of morphine. Clonidine and sulfisoxazole when administered together produced more than 167% increase in AUC compared either clonidine or sulfisoxazole administered alone. These findings confirmed that non-opiates, clonidine and sulfisoxazole, administered together can produce marked antinociception which is comparable to morphine (FIG. 4).

### Example 6

### Dose Response Effect of Clonidine on Analgesia

To determine the dose effect response of clonidine on analgesia, mice were administered clonidine at 0.3 mg/ml, 1.0 mg/ml and 3.0 mg/ml and the effects on analgesia were measured.

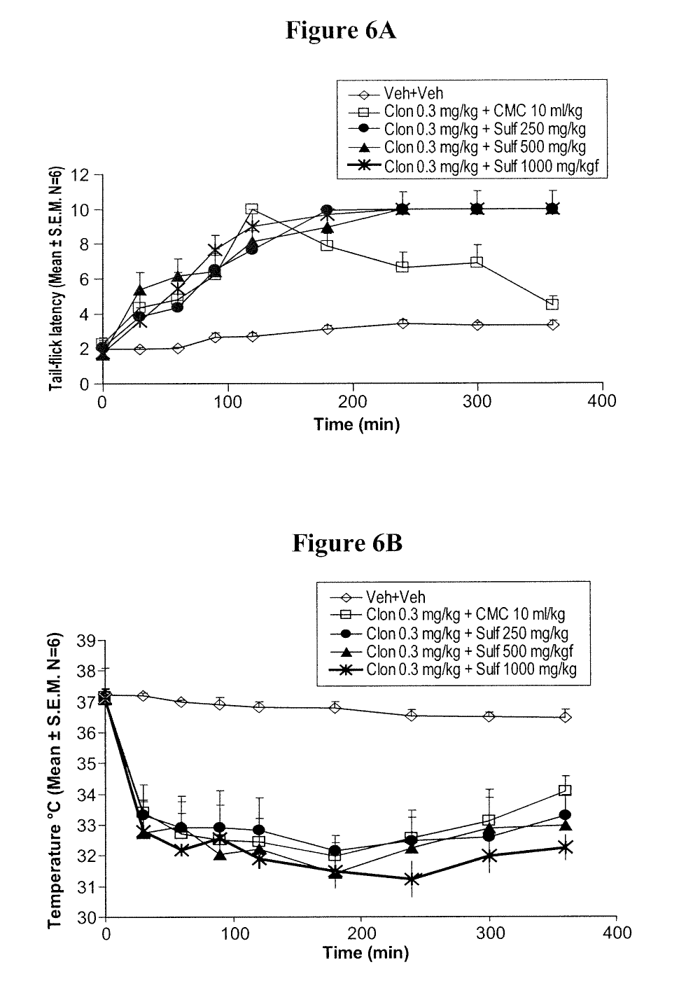

FIGS. 5A and 5B illustrate the dose response effect of clonidine on analgesia (tail flick latency) and body temperature in mice administered either vehicle (saline), clonidine at 0.3, 1.0 or 3.0 mg/kg. Results show that clonidine at 3.0 mg/kg increased tail flick latency to approximately 5-fold over vehicle for all time points measured. Clonidine at 1.0 mg/kg increased tail-flick latency to approximately 5-fold up to approximately 3 hours after injury, but fell to 3-fold over vehicle at approximately 6 hours. Clonidine at 0.3 mg/ml surprisingly increased tail-flick latency above vehicle up to 4-fold immediately after injury, decreasing to approximately 3-fold at 3 hours, and decreasing to 2.5-fold by 5 hours. These results demonstrate that the effects of clonidine on analgesia are dose dependent, and a dose of clonidine as low as 0.3 mg/kg was effective at producing analgesia.

Dose response studies show that all doses of clonidine decreased body temperature of treated animals in a similar manner (FIG. 5B).

### Example 7

### Dose Response Effects of Clonidine and Sulfisoxizole on Analgesia

As shown above, clonidine demonstrates a dose response effect on analgesia in treated animals. To determine if addition of sulfisoxazole to the treatment regimen also produced a dose response effect, clonidine at 0.3 mg/kg was administered with varying doses of sulfisoxazole.

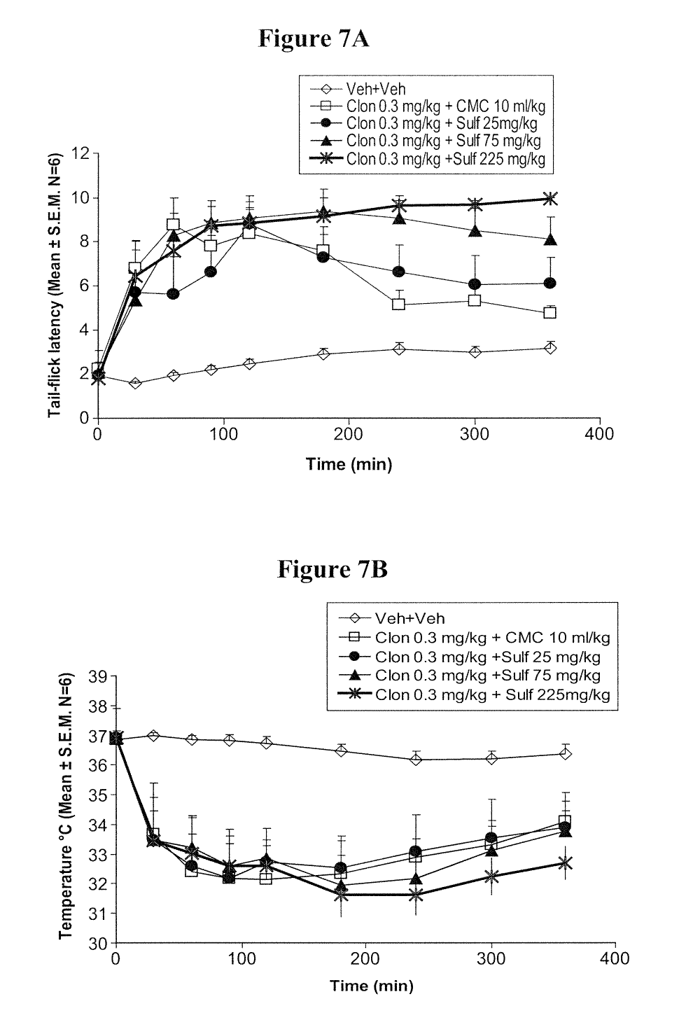

Mice were treated with 0.3 mg/kg clonidine and either 0.5% CMC (10 ml/kg) or sulfisoxazole at 250, 500 or 1000 mg/kg. Results show that clonidine with 0.5% CMC or any dose of sulfisoxazole reduced body temperature in a similar manner, from 37° C. to as low as approximately 32° C. after 3 hours (FIG. 6B), and decreased body temperature comparable to all doses of clonidine seen in FIG. 5B. In the tail flick latency test for analgesia, clonidine at 0.3 mg/kg plus CMC increased tail flick latency by 5-fold at 2 hours post injury, but decreased to approximately the level of vehicle treated animals by 6 hours. The effects of sulfisoxazole on clonidine induced analgesia were not dose dependent (FIG. 6A). Sulfisoxazole at any of 250, 500 or 1000 mg/kg administered with clonidine at 0.3 mg/kg increased tail-flick latency up to approximately 5-fold by 2 hours after injury, and maintained this level of analgesia by 6 hours after injury.

These results show that the analgesic effect of clonidine was significantly potentiated by sulfisoxazole, even at the lowest dose of sulfisoxazole (250 mg/kg).

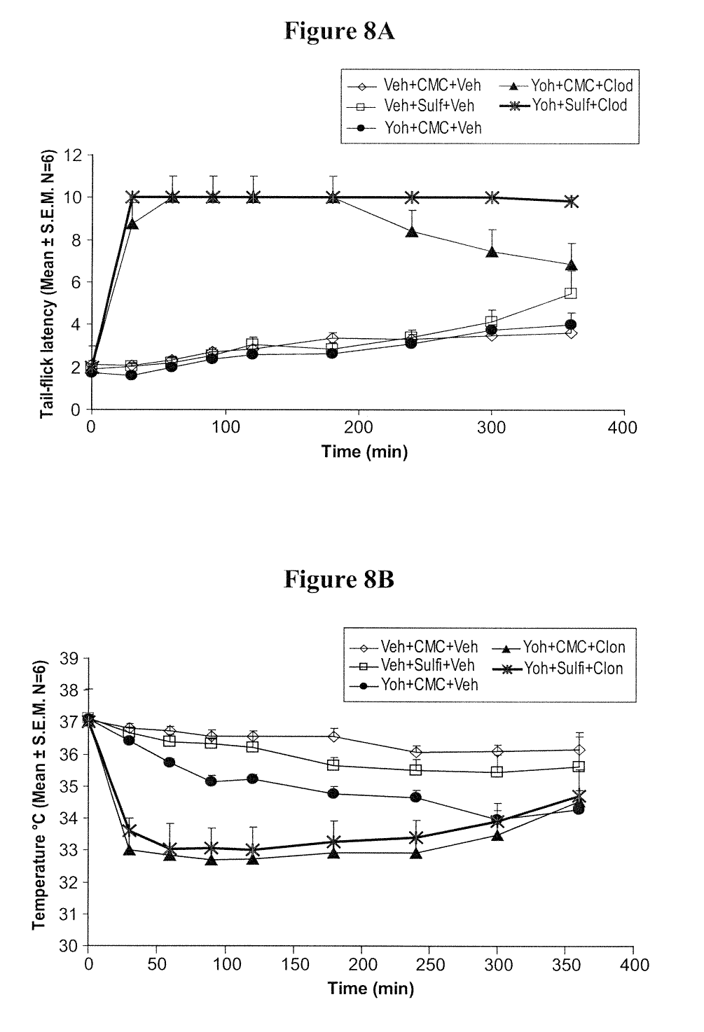

Additional studies were carried out to determine the lowest dose of sulfisoxazole that can potentiate the analgesic effects of clonidine. Mice were administered clonidine (0.3 mg/kg) and either 0.5% CMC, or sulfisoxazole at 25, 75 or 225 mg/kg. Results show that sulfisoxazole had no effect on clonidine-induced decrease in body temperature (FIG. 7B), but that sulfisoxazole does have a dose dependent effect on clonidine-induced analgesia (FIG. 7A). Sulfisoxazole at 225 mg/kg increased tail-flick latency by 4- to 5-fold by 1.5 hours post injury, and maintained this level of analgesia by 6 hours after injury. Sulfisoxazole at 75 mg/kg increased tail-flick latency approximately 4-fold up to 3 hours, but the analgesic effects decreased by 6 hours to approximately 3-fold over the tail flick latency of vehicle treated animals. Sulfisoxazole at 25 mg/kg potentiated clonidine-induced analgesia to approximately 5-fold greater than vehicle treated mice, but this level decreased over time to approximately 2-fold greater than vehicle by 6 hours post treatment. Varying doses of sulfisoxazole had no effect on body temperature.

These results show that low doses of sulfisoxazole (225 and 75 mg/kg) potentiate the analgesic effects of low dose clonidine (0.3 mg/kg). Thus, the combination of the two agents produces an analgesic effect similar to that of morphine, but at lower doses than is normally seen with clonidine alone or sulfisoxazole alone when given at higher doses.

### Example 8

### Mechanism of Clonidine Analgesia

To determine the mechanism of action of clonidine and sulfisoxazole on inducing analgesia, clonidine was administered with several different agents that effect different pain receptors, e.g., alpha-2 adrenergic receptors, imidazoline adrenergic receptors or opioid receptors.

To determine if α₂ adrenergic receptors mediate clonidine analgesia, yohimbine, an α₂ adrenergic receptor antagonist, was administered in combination with clonidine plus CMC or clonidine plus sulfisoxazole and tail flick latency and body temperature were measured. Mice were divided into 5 groups, Group 1: Vehicle+vehicle (saline i.p.)+vehicle (saline, i.p.); Group 2 Vehicle+CMC (p.o.)+vehicle (saline, i.p.); Group 3: Yohimbine (2 mg/kg, ip)+CMC (p.o.)+vehicle (saline, i.p.); Group 4: Yohimbine (2 mg/kg, ip)+CMC (p.o.)+Clonidine (0.3 mg/kg, i.p.); Group 5: Yohimbine (2 mg/kg, ip)+sulfisoxazole (250 mg kg, p.o.)+Clonidine (0.3 mg/kg, i.p.).

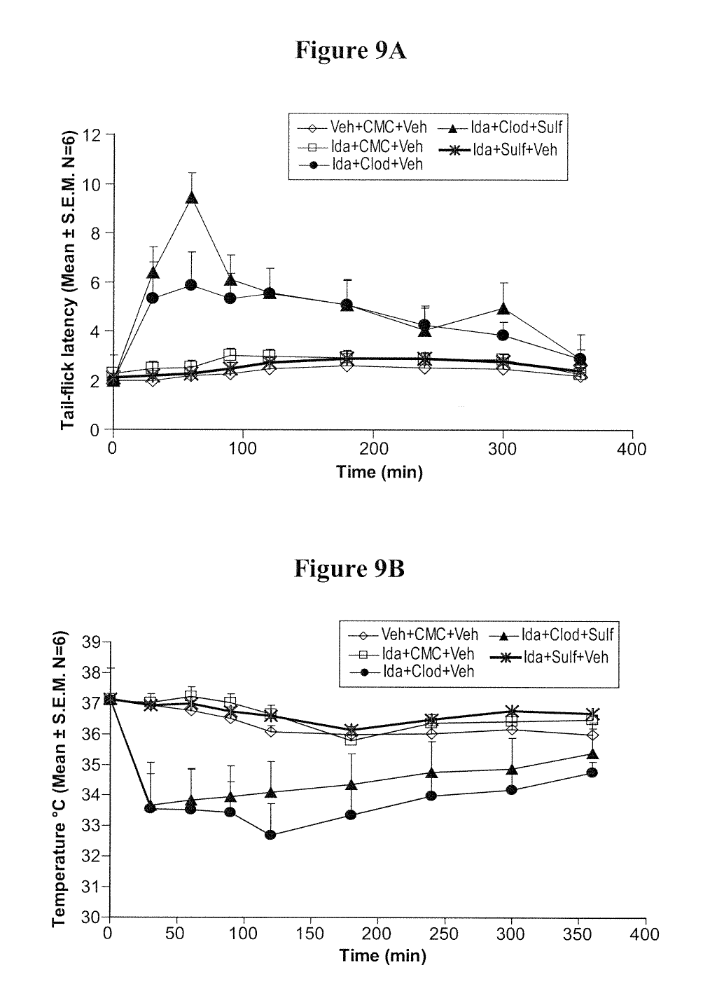

Yohimbine partially reduced the fall in temperature induced by clonidine (FIG. 8B). Administration of yohimbine, clonidine and sulfisoxazole shows that yohimbine did not affect the analgesic effect of clonidine plus sulfisoxazole (FIG. 8A). These studies indicate that the analgesic effect of clonidine plus sulfisoxazole is not mediated through α₂ adrenergic receptors.

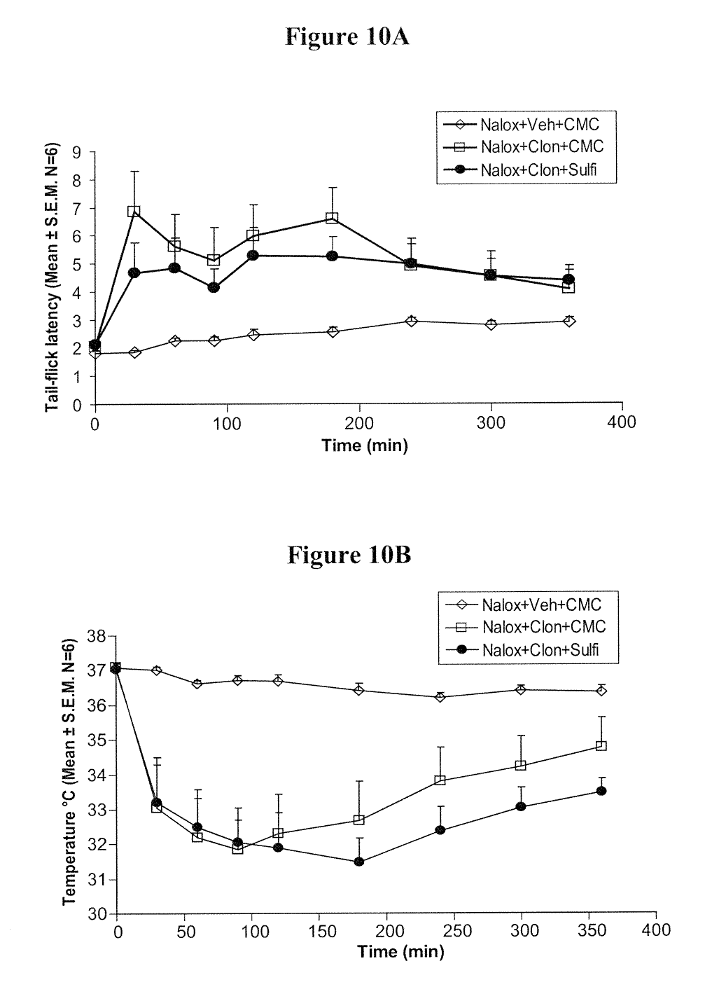

To determine if imidazoline adrenergic receptors mediate clonidine analgesia, idazoxan, an imidazoline receptor antagonist, was administered in combination with clonidine plus CMC or clonidine plus sulfisoxazole and tail flick latency and body temperature were measured. Mice were divided into 5 groups, Group 1: Vehicle+Vehicle (saline i.p.)+vehicle (CMC), p.o.); Group 2 Idazoxan (2 mg/kg, ip)+CMC (p.o.)+vehicle (saline, i.p.); Group 3: Idazoxan (2 mg/kg, ip)+CMC (p.o.)+vehicle (saline i.p.); Group 4: Idazoxan (2 mg/kg, ip)+CMC (p.o.)+Clonidine (0.3 mg/kg, i.p.); Group 5: Idazoxan (2 mg/kg, ip)+sulfisoxazole (250 mg/kg, p.o.)+Clonidine (0.3 mg/kg, i.p.). Idazoxan partially reduced the fall in temperature induced by clonidine (FIG. 9B). Administration of idazoxan to mice receiving clonidine and sulfisoxazole reduced the analgesic effect of clonidine plus sulfisoxazole (FIG. 9A). These studies indicate that the analgesic effect of clonidine plus sulfisoxazole may in part be mediated through imidazoline adrenergic receptors.

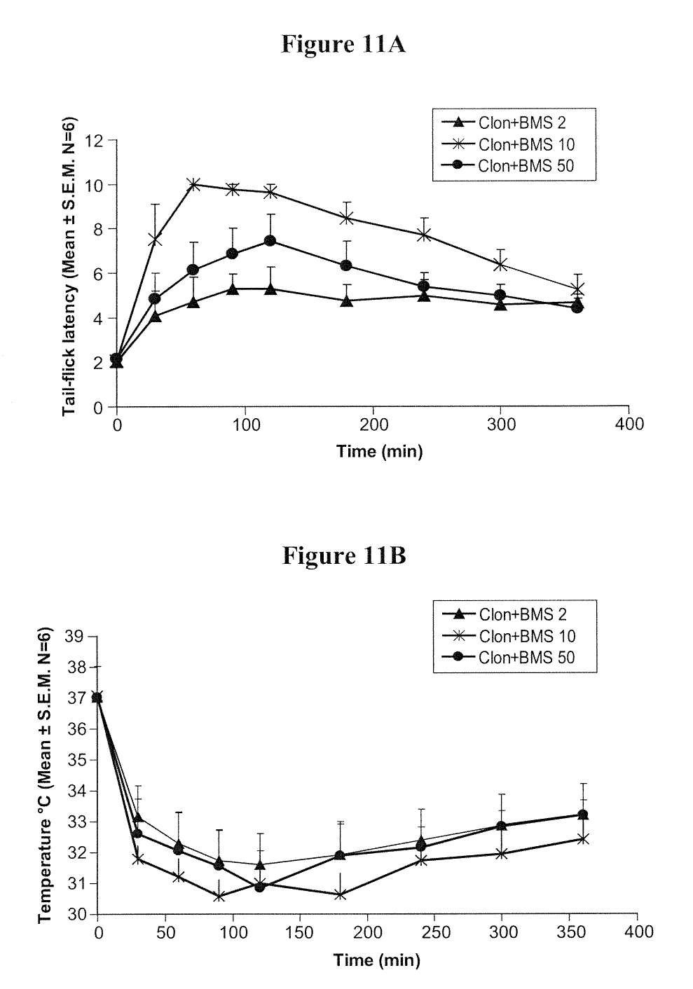

To determine if opioid adrenergic receptors mediate clonidine analgesia, naloxone, an opioid receptor antagonist, was administered in combination with clonidine plus CMC or clonidine plus sulfisoxazole and tail flick latency and body temperature were measured. Mice were divided into 5 groups, Group 1: Naloxone (1.0 mg/kg, i.p.)+CMC (p.o.)+vehicle (saline i.p.); Group 2: Naloxone (1.0 mg/kg, i.p.)+CMC (p.o.)+Clonidine (0.3 mg/kg, i.p.); Group 3: Naloxone (1.0 mg/kg, i.p.)+sulfisoxazole (250 mg/kg, p.o.)+Clonidine (0.3 mg/kg, i.p.). Naloxone but did not affect the fall in temperature induced by clonidine (FIG. 10B). However, administration of naloxone reduced the analgesic effect of clonidine plus sulfisoxazole, from approximately 5-fold increase in tail-flick latency (see FIG. 5A) to only 2-fold increase in tail flick latency at 4 hours post treatment (FIG. 10A). These studies indicate that the analgesic effect of clonidine plus sulfisoxazole is mediated through opioid receptors.

### Example 9

### Effect of Other Endothelin A Antagonist on Clonidine Induced Analgesia

To determine if the effect of sulfisoxazole on clonidine analgesia is specific to the molecule or if additional ET_{A} antagonists also potentiate cloinidine analgesia, tail flick latency and body temperature were measured in mice administered clonidine and the ET_{A} antagonist BMS182874.

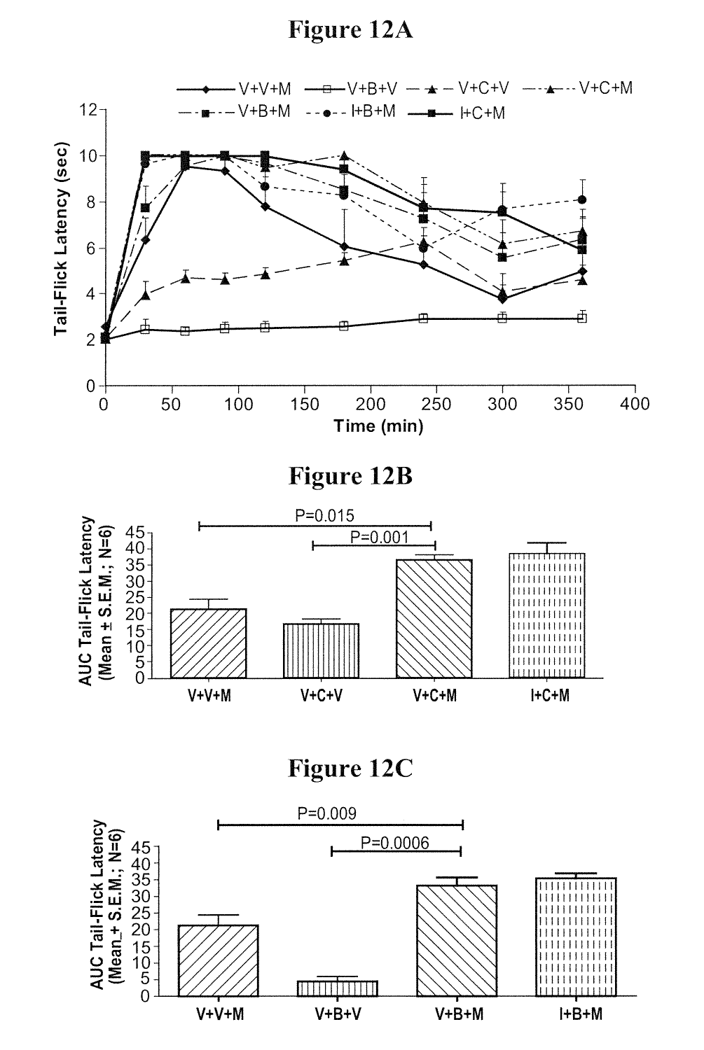

Mice were divided into three treatment groups, Group 1: Clonidine (0.3 mg/kg, i.p.)+BMS 182874 (2.0 μg/kg, icv); Group 2: Clonidine (0.3 mg/kg, i.p.)+BMS 182874 (10.0 μg/kg, icv); Group 3: Clonidine (0.3 mg/kg, i.p.)+BMS 182874 (50.0 μg/kg, icv). It was found that the low and high dose of BMS182874 moderately potentiated clonidine analgesia but the middle dose (10 μg/kg) potentiated the analgesic effect of clonidine (FIG. 11A), increasing the tail flick latency 5-fold at 1 and 2 hours post treatment, decreasing to approximately 3-fold at 4 hours post treatment and 2-fold by 6 hours post treatment (FIG. 11A compared to vehicle results in FIG. 5A). BMS182874 did not affect the fall in temperature induced by clonidine (FIG. 11B).

These results indicate that ET_{A} receptor may be involved in the potentiation of analgesic effect of clonidine, and that different ET_{A} antagonists, used at an optimized dose, can potentiate clonidine-induced analgesia.

### Example 10

### Effect of Clonidine or BMS182874 on Opioid Analgesia

Differences between morphine and oxycodone analgesia have been reported [40,41,42]. Oxycodone has been clinically used since 1917 [43]. It is a μ-opioid receptor agonist [44] with Ki value of 18 nM for μ-opioid receptors, 958 nM for δ-opioid receptors and 677 nM for K-opioid receptors [45]. However, oxycodone has >20 times less affinity to the μ-opioid receptors than morphine. Further evidence of differences between analgesic action of morphine and oxycodone comes from the finding that morphine has greater analgesic action in male compared to female rats, whereas, oxycodone has a similar analgesic action in both male and female rats [46]. Idazoxan, an α₂ adrenergic receptor inhibitor, has a Ki value of 3.6 nM at α₂-adrenergic receptors and 186 nM at 11-imidazoline receptors [47] indicating that idazoxan acts on α₂-adrenergic receptors and therefore, should block the potentiation of analgesic action of morphine by clonidine. Clonidine has been shown to have Ki values of 3.8 nM at α₂-adrenergic receptor sites and 1.0 nM at I₁-imidazoline receptors [48].

In order to determine the effects of clonidine and additional ET_{A} antagonists on potentiation of opioid analgesia, tail flick latency of mice administered clonidine, BMS182874 and an opioid was assessed in the absence or presence of idazoxan.

Effect of Clonidine or BMS182874 on Morphine Analgesia and its Blockade by Idazoxan

Subject animals ere divided into the following groups (n=6 per group): Group 1: Vehicle (1 ml/kg, ip)+vehicle (5 μl, icv)+morphine (8 mg/kg, sc), Group 2: Vehicle (1 ml/kg, ip)+clonidine (1 mg/kg, ip)+vehicle (1 ml/kg, sc), Group 3: Vehicle (1 ml/kg, ip)+clonidine (1 mg/kg, ip)+morphine (8 mg/kg, sc), Group 4: Idazoxan (2 mg/kg, ip)+clonidine (1 mg/kg, ip)+morphine (8 mg/kg, sc), Group 5: Vehicle (1 ml/kg, ip)+vehicle (5 μl, icv)+morphine (8 mg/kg, sc), Group 6: Vehicle (1 ml/kg, ip)+BMS182874 (50 μg, icv)+vehicle (1 ml/kg, sc), Group 7: Vehicle (1 ml/kg, ip)+BMS182874 (50 μg, icv)+morphine (8 mg/kg, sc), Group 8: Idazoxan (2 mg/kg, ip)+BMS182874 (50 μg, icv)+morphine (8 mg/kg, sc).

Morphine sulfate, 7,8-didehydro-4,5α-epoxy-17-methylmorphinan-3,6α-diol sulfate (Mallinckrodt Chemical Co., St. Louis, Mo.) was dissolved in sterile saline and injected subcutaneously (sc). Clonidine, N-(2,6-dichlorophenyl)-4,5-dihydro-1H-imidazol-2-amine (Sigma Chemical Company, St. Louis, Mo.) was dissolved in sterile saline and injected intraperitoneally (ip). BMS182874, 5-(dimethylamino)-N-(3,4-dimethyl-5-isoxazolyl)-1-naphthalene sulfonamide (Tocris Pharmaceuticals Inc., Ellisville, Mo.) was dissolved in 20% DMSO and injected intracerebroventricularly (i.c.v.). Idazoxan, 2-(1,4-benzodioxan-2-yl)-2-imidazoline (Sigma Chemical Company, St. Louis, Mo.) was dissolved in sterile saline and injected intraperitoneally (ip). For the studies, idazoxan was administered 15 min before clonidine or BMS182874 treatment; morphine was administered 30 min after clonidine or BMS182874 treatment.

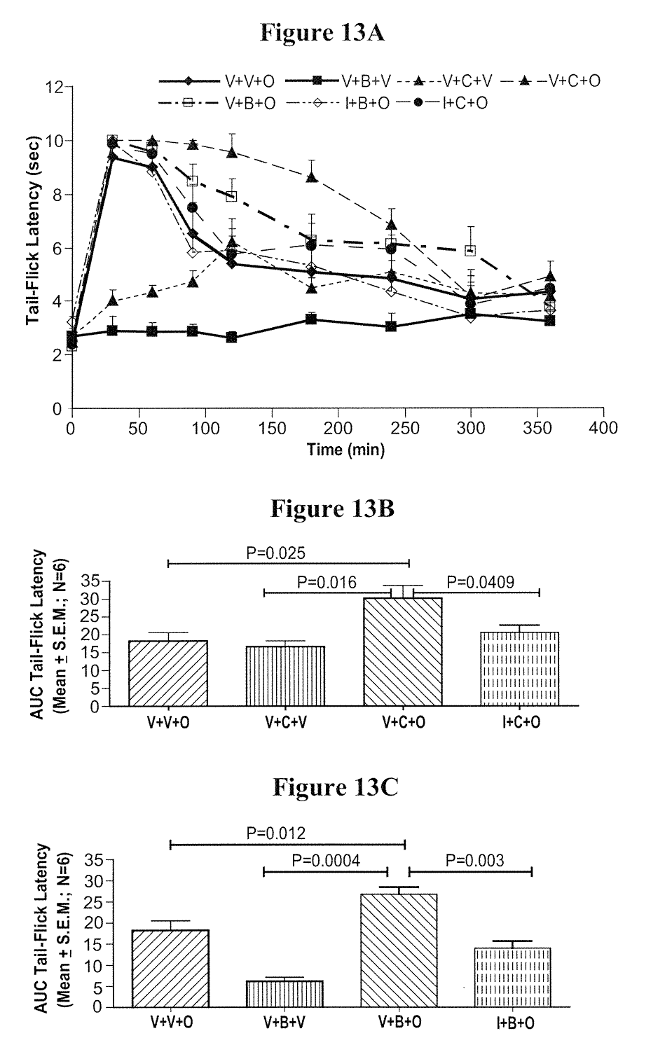

Morphine (8 mg/kg, sc) produced significant analgesia with an AUC_{0→360} of 21.23±3.18 sec.min (FIG. 12B). Clonidine (1 mg/kg, ip) also produced analgesia with an AUC_{0→360} of 16.62±1.61 sec.min (FIG. 12B). In rats treated with clonidine (1 mg/kg, ip) plus morphine (8 mg/kg, sc) a significantly greater analgesia was produced compared to rats treated with morphine (P=0.015) or clonidine (P=0.001) alone. Idazoxan (2 mg/kg, ip) did not affect analgesia produced by clonidine plus morphine in rats (FIG. 12A).

BMS182874 (50 μg, icv) alone did not produce any analgesic effect, yielding an AUC_{0→360} of 4.47±1.49 sec.min. However, rats treated with BMS182874 (50 μg, icv) plus morphine (8 mg/kg, sc) exhibited a significantly greater analgesia compared to rats treated with morphine (P=0.009) or BMS182874 (P=0.0006) alone (FIG. 12C). Idazoxan (2 mg/kg, ip) did not affect analgesia produced by BMS182874 plus morphine in rats (FIG. 12A). The results showed that clonidine (P=0.015) and BMS182874 (P=0.009) significantly potentiated morphine analgesia.

Effect of clonidine or BMS182874 on oxycodone analgesia and its blockade by idazoxan: In experiments to assess the effects of clonidine of BMS182874 on oxycodone analgesia, treatment groups were divided as follows (n=6 per group): Group 1: Vehicle (1 ml/kg, ip)+vehicle (1 ml/kg, ip)+oxycodone (4 mg/kg, sc), Group 2: Vehicle (1 ml/kg, ip)+clonidine (1 mg/kg, ip)+vehicle (1 ml/kg, sc), Group 3: Vehicle (1 ml/kg, ip)+clonidine (1 mg/kg, ip)+oxycodone (4 mg/kg, sc), Group 4: Idazoxan (2 mg/kg, ip)+clonidine (1 mg/kg, ip)+oxycodone (4 mg/kg, sc), Group 5: Vehicle (1 ml/kg, ip)+vehicle (5 μl, icv)+oxycodone (4 mg/kg, sc), Group 6: Vehicle (1 ml/kg, ip)+BMS182874 (50 μg, icv)+vehicle (1 ml/kg, sc), Group 7: Vehicle (1 ml/kg, ip)+BMS182874 (50 μg, icv)+oxycodone (4 mg/kg, sc), Group 8: Idazoxan (2 mg/kg, ip)+BMS182874 (50 μg, icv)+oxycodone (4 mg/kg, sc).

Clonidine. BMS182874 and idazoxan were prepared as above. Oxycodone hydrochloride, 4,5α-epoxy-14-hydroxy-3-methoxy-17-methylmorphinan-6-one hydrochloride (Spectrum Chemicals, Inc., San Gardena, Calif.) was dissolved in sterile saline and injected subcutaneously (sc). Oxycodone was administered 30 min after clonidine or BMS182874 treatment. Idazoxan was given 15 min before clonidine or BMS182874 treatment.

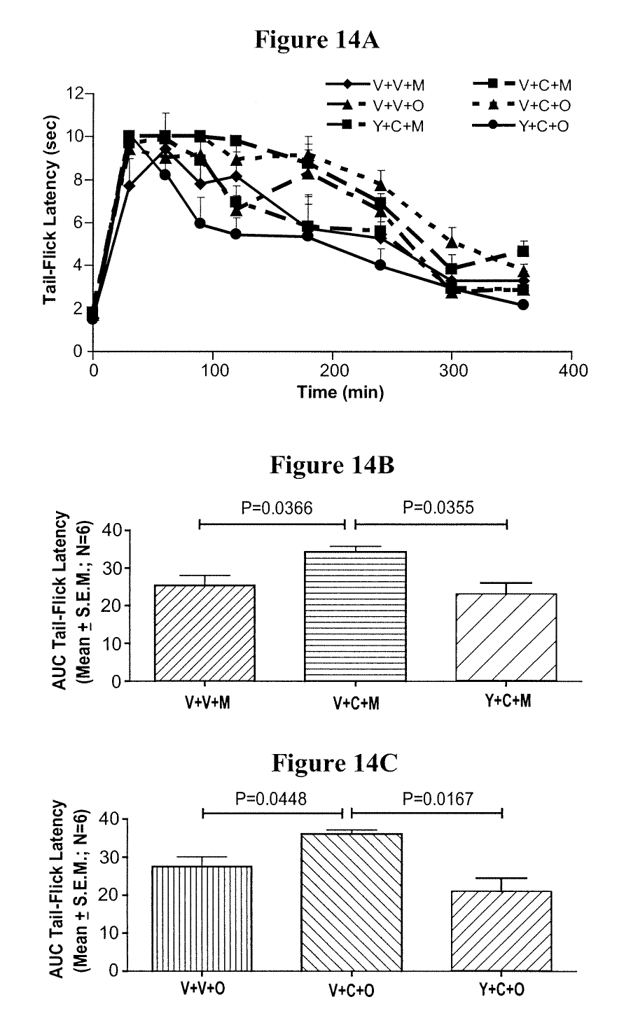

Results showed that oxycodone (4 mg/kg, sc) produced significant analgesia with an AUC_{0→360} of 18.23±2.32 sec.min. Clonidine (1 mg/kg, ip) also produced analgesia. In rats treated with clonidine (1 mg/kg, ip) plus oxycodone (4 mg/kg, sc) a significantly greater analgesia was observed compared to rats that received oxycodone (P=0.025) or clonidine (P=0.016) alone (FIG. 13B). Idazoxan (2 mg/kg, ip) blocked the analgesia produced by clonidine plus oxycodone in rats (FIG. 13A).

Oxycodone (4 mg/kg, sc) produced significant analgesia while BMS182874 (50 μg, icv) did not produce any analgesic effect (FIG. 13A). However, in rats treated with BMS182874 (50 μg, icv) plus oxycodone (4 mg/kg, sc) a significantly greater analgesia was produced compared to rats in which oxycodone (P=0.012) or BMS182874 (P=0.0004) alone was administered (FIG. 13C). Idazoxan (2 mg/kg, ip) significantly (P=0.003) blocked the analgesic effect produced by BMS182874 plus oxycodone in rats (FIG. 13A,13C). These results showed that clonidine (P=0.025) and BMS182874 (P=0.0 12) potentiated oxycodone analgesia.

Overall, idazoxan, an I₁-imidazoline and α₂ adrenergic receptor antagonist, was found to block the potentiation of oxycodone analgesia by clonidine or BMS182874, but idazoxan did not affect the potentiation of morphine analgesia by clonidine or BMS182874. This finding indicates the involvement of imidazoline receptors in oxycodone analgesia—I₁-imidazoline receptors appear to be involved in potentiation of oxycodone analgesia but not in potentiation of morphine analgesia by clonidine or BMS182874. This is the first report describing that clonidine or BMS182874 induced potentiation of oxycodone analgesia involves I₁-imidazoline receptors, whereas, morphine analgesia does not.

### Example 11

### Effect of Yohimbine on Clonidine Induced Increase in Morphine or Oxycodone Analgesia

In order to determine the involvement of α₂-adrenergic receptor a selective antagonist, yohimbine, was used. Yohimbine is a highly selective α₂-adrenergic receptor antagonist with Ki values of 22 nM for α₂-adrenergic receptors and 21,810 nM for I₁-imidazoline receptors [49]. Yohimbine does not bear an imidazoline ring and it does not act on imidazoline receptors.

Treatment groups were divided as follows (n=6 per group): Group 1: Vehicle (1 ml/kg, ip)+vehicle (5 μl, icv)+morphine (4 mg/kg, sc), Group 2: Vehicle (1 ml/kg, ip)+BMS182874 (50 μg, icv)+morphine (4 mg/kg, sc), Group 3: Yohimbine (2 mg/kg, ip)+BMS182874 (50 μg, icv)+morphine (4 mg/kg, sc), Group 4: Vehicle (1 ml/kg, ip)+vehicle (5 μl, icv)+oxycodone (4 mg/kg, sc), Group 5: Vehicle (1 ml/kg, ip)+BMS182874 (50 μg, icv)+oxycodone (4 mg/kg, sc), Group 6: Yohimbine (2 mg/kg, ip)+BMS182874 (50 μg, icv)+oxycodone (4 mg/kg, sc)

Yohimbine hydrochloride, 17α-hydroxy-yohimban-16α-carboxylate hydrochloride (Sigma Chemical Company, St. Louis, Mo.) was dissolved in alcohol (1 part) and sterile saline (9 parts) and injected intraperitoneally (ip). Yohimbine was administered 15 min before BMS182874 treatment; and morphine or oxycodone was administered 30 min after BMS182874 treatment.

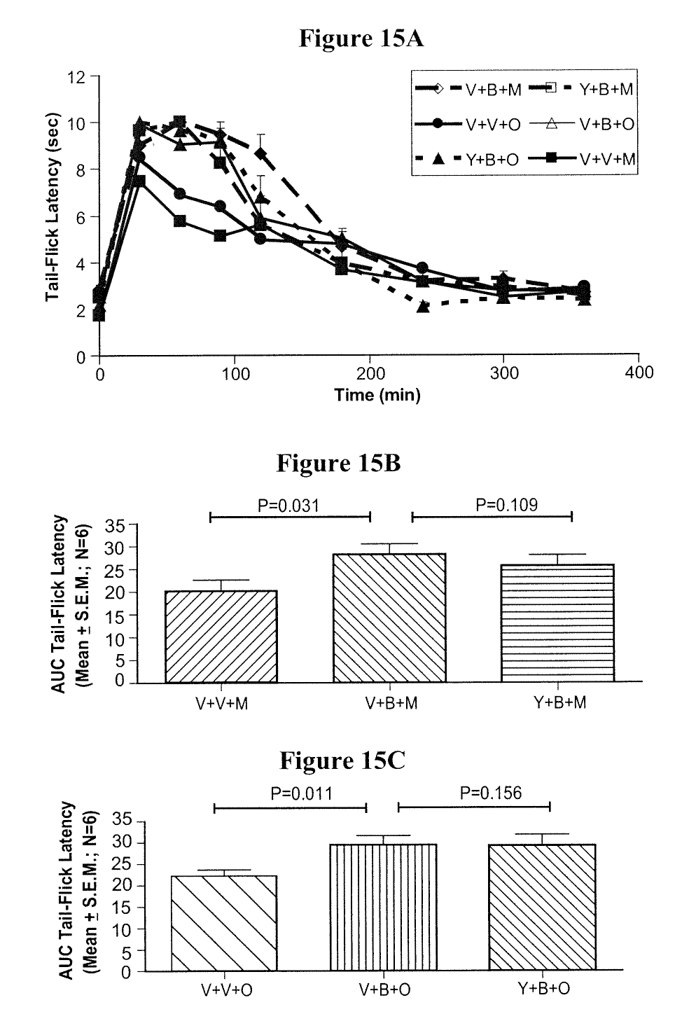

Results show that clonidine potentiated morphine (P=0.0366), as well as, oxycodone (P=0.0587) analgesia, and yohimbine blocked clonidine induced potentiation of analgesic effect of morphine (P=0.058) or oxycodone (P=0.0167) (FIGS. 14A-14C). The antagonism was borderline significant in morphine but highly significant in oxycodone analgesia. The increase in morphine analgesia (8 mg/kg, sc) was attenuated (P=0.0355) by yohimbine pretreatment (FIG. 14B). Similarly, it was found that clonidine increased analgesia induced by oxycodone (4 mg/kg, sc), which was significantly blocked by yohimbine pretreatment (FIG. 14C).

In order to determine the involvement of α₂ adrenergic receptor in BMS182874 induced increase in morphine and oxycodone analgesia, the α₂ adrenergic selective antagonist yohimbine was administered to animals receiving the agents. Treatment groups were divided as follows (n=6 per group): Group 1: Vehicle (1 ml/kg, ip)+vehicle (5 μl, icv)+morphine (4 mg/kg, sc), Group 2: Vehicle (1 ml/kg, ip)+BMS182874 (50 μg, icv)+morphine (4 mg/kg, sc), Group 3: Yohimbine (2 mg/kg, ip)+BMS182874 (50 μg, icv)+morphine (4 mg/kg, sc), Group 4: Vehicle (1 ml/kg, ip)+vehicle (5 μl, icv)+oxycodone (4 mg/kg, sc), Group 5: Vehicle (1 ml/kg, ip)+BMS182874 (50 μg, icv)+oxycodone (4 mg/kg, sc), Group 6: Yohimbine (2 mg/kg, ip)+BMS182874 (50 μg, icv)+oxycodone (4 mg/kg, sc).

Yohimbine was administered 15 min before BMS182874 treatment; morphine or oxycodone was administered 30 min after BMS182874 treatment.

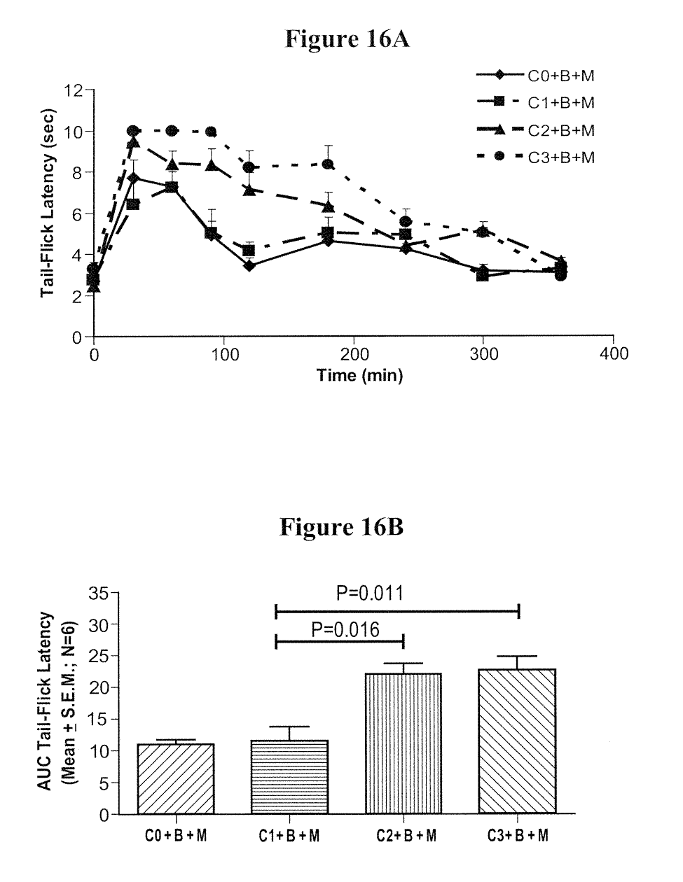

As shown above, BMS182874 potentiates (P=0.000 1) analgesia induced by oxycodone (4 mg/kg, sc), and increases (P=0.003) analgesia induced by morphine (8 mg/kg, sc) (FIGS. 15A and 15B). BMS182874 potentiated morphine (P=0.003), and oxycodone (P=0.0001) analgesia (FIGS. 15B and 15C). Administration of yohimbine to BMS182874 treated animal showed that analgesia produced by morphine or oxycodone in rats treated with BMS182874 was not affected (P=0.156) by yohimbine pretreatment (FIG. 15C).

These results show that potentiation of morphine and oxycodone analgesia by clonidine was blocked by yohimbine, a selective α₂ adrenergic antagonist, thereby indicating α₂ adrenergic receptors are involved in the augmentation of morphine and oxycodone analgesia by clonidine. These findings also indicate that the potentiation of morphine and oxycodone analgesia by BMS182874 and clonidine are through different mechanisms.

### Example 12

### Dose-Response Effect of Clonidine on Morphine and Oxycodone Analgesia in BMS182874 Treated Rats

As seen above, the dose of clonidine or sulfisoxazole correlated with the levels of analgesia seen in treated animals. To determine the dose effect of clonidine and ET_{A} antagonist BMS182874 on opioid analgesia, a dose response study was carried out.

Treatment groups were divided as follows (n=6 per group): Group 1: Vehicle (1 ml/kg, ip)+BMS182874 (50 μg, icv)+morphine (4 mg/kg, sc), Group 2: Clonidine (0.1 mg kg, ip)+BMS182874 (50 μg, icv)+morphine (4 mg/kg, sc), Group 3: Clonidine (0.3 mg/kg, ip)+BMS182874 (50 μg, icv)+morphine (4 mg/kg, sc), Group 4: Clonidine (1.0 mg/kg, ip)+BMS182874 (50 μg, icv)+morphine (4 mg/kg, sc), Group 5: Vehicle (1 ml/kg, ip)+BMS182874 (50 μg, icv)+oxycodone (4 mg/kg, sc), Group 6: Clonidine (0.1 mg/kg, ip)+BMS182874 (50 μg, icv)+oxycodone (4 mg/kg, sc), Group 7: Clonidine (0.3 mg/kg, ip)+BMS182874 (50 μg, icv)+oxycodone (4 mg/kg, sc), Group 8: Clonidine (1.0 mg/kg, ip)+BMS182874 (50 μg, icv)+oxycodone (4 mg/kg, sc).

Clonidine was administered 15 min before BMS182874 treatment, and either morphine or oxycodone was administered 30 min after BMS182874 treatment. Other active agents were prepared and administered as described above.

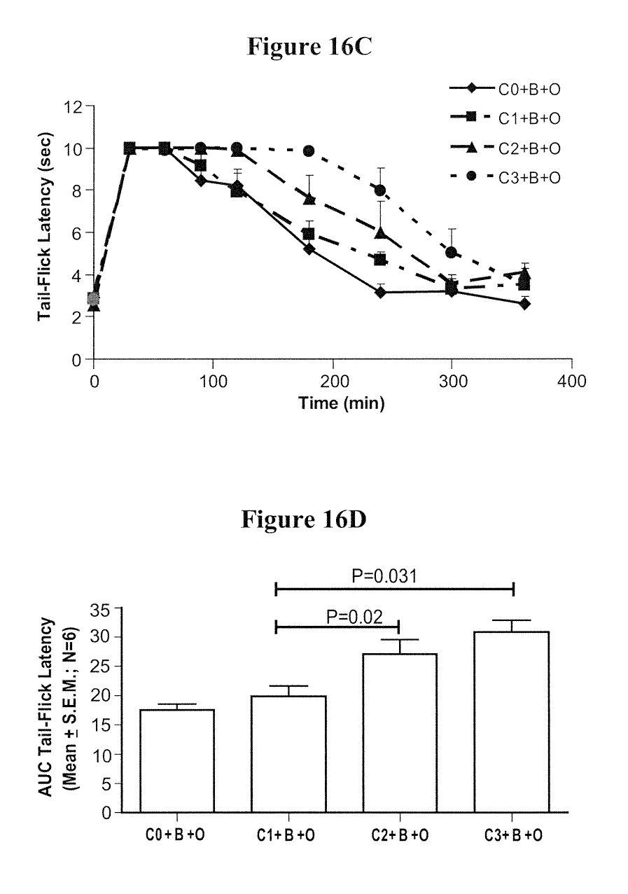

Morphine (4 mg/kg, sc) produced significant analgesia in rats treated with BMS182874. Clonidine produced a dose-dependent increase in analgesic effect of morphine in rats treated with BMS182874 (FIG. 16A). Clonidine 0.1 mg/kg, ip dose did not produce an increase in analgesic effect, however 0.3 and 1.0 mg/kg, ip dose of clonidine produced a significant (P=0.016 and P=0.011, respectively) increase in morphine analgesia in rats treated with BMS182874 (FIGS. 16A and 16B).

Oxycodone (4 mg/kg, sc) produced significant analgesia in rats treated with BMS182874. Clonidine produced a dose-dependent increase in analgesic effect of oxycodone in rats treated with BMS182874 (FIG. 16C). Clonidine 0.1 mg/kg, ip dose did not produce an increase in analgesic effect, however 0.3 and 1.0 mg/kg, ip dose of clonidine produced a significant (P=0.02 and P=0.031, respectively) increase in oxycodone analgesia in rats treated with BMS182874 (FIGS. 16C and 16D).

### Example 13

### Effect of Clonidine, BMS182874 and Clonidine Plus BMS182874 on Opioid Analgesia

In order to confirm an increase in analgesic activity of morphine or oxycodone by combined use of clonidine and BMS182874, the effect of clonidine (1 mg/kg, ip), BMS182874 (50 μg, icv) and clonidine (1 mg kg, ip) plus BMS182874 (50 μg, icv) in morphine (8 mg/kg, sc) and oxycodone (4 mg/kg, sc) treated rats was determined.

Treatment groups were divided as follows (n=6 per group): Group 1: Vehicle (1 ml/kg, ip)+clonidine (1 mg/kg, ip)+morphine (8 mg/kg, sc), Group 2: Vehicle (1 ml/kg, ip)+BMS182874 (50 μg, icv)+morphine (8 mg/kg, sc), Group 3: Clonidine (1.0 mg/kg, ip)+BMS182874 (50 μg, icv)+morphine (8 mg kg, sc), Group 4: Vehicle (1 ml/kg, ip)+clonidine (1 mg/kg, ip)+oxycodone (4 mg/kg, sc), Group 5: Vehicle (1 ml/kg, ip)+BMS182874 (50 μg, icv)+oxycodone (4 mg/kg, sc), Group 6: Clonidine (1.0 mg/kg, ip)+BMS182874 (50 μg, icv)+oxycodone (4 mg/kg, sc).

Clonidine was administered 15 min before BMS182874 treatment. Morphine or oxycodone was administered 30 min after BMS182874 or clonidine treatment.

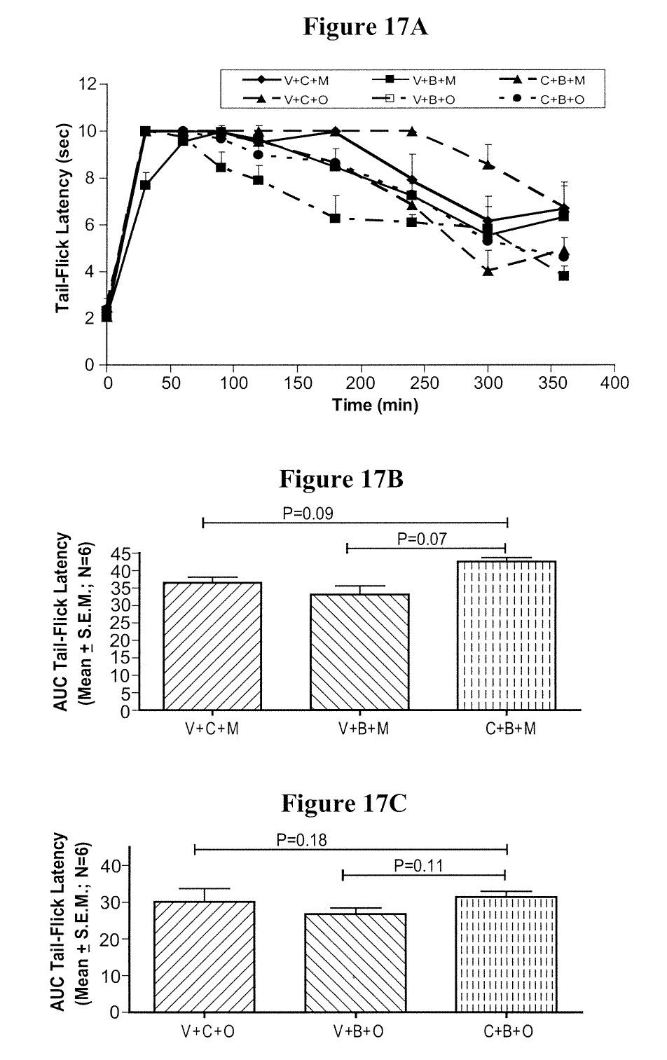

Results showed there was no further increase in analgesic activity of morphine in clonidine plus BMS182874 treated rats (FIG. 17A) compared to either clonidine (P=0.09) or BMS182874 (P=0.07) alone (FIG. 17B). Similarly, it was found that there was no further increase in analgesic activity of oxycodone in clonidine plus BMS182874 treated rats compared to either clonidine (P=0. 18) or BMS182874 (P=0.11) alone (FIG. 17C).

### Example 14

### Effect of BMS182874 on Clonidine Analgesia

In order to determine if analgesic activity of clonidine (1 mg/kg, ip) is increased by BMS182874 (50 μg, icv), the effect of BMS182874, clonidine and clonidine plus BMS182874 was determined in rats.

Treatment groups were divided as follows (n=6 per group): Group 1: BMS182874 (50 μg, icv)+vehicle (1 ml/kg, ip), Group 2: Vehicle (5 μl, icv)+clonidine (1.0 mg/kg, ip), Group 3: BMS182874 (50 μg, icv)+clonidine (1.0 mg/kg, ip). BMS182874 was administered 15 min before clonidine treatment.

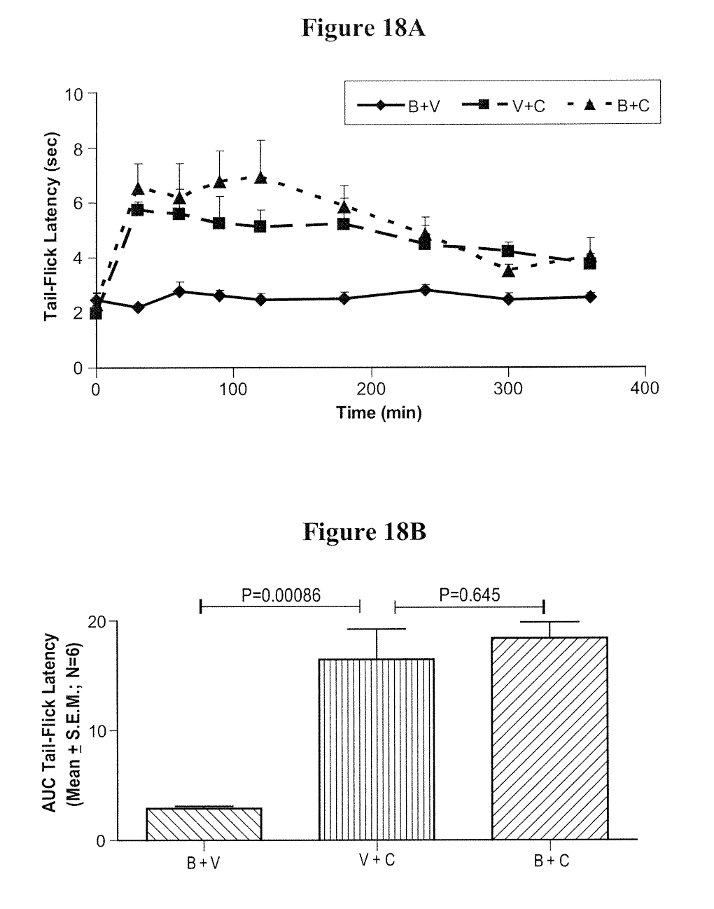

Results showed that BMS182874 did not produce any analgesic effect, whereas clonidine produced significant (P=0.00086) analgesia compared to BMS182874 (FIG. 18A). However, BMS182874 did not affect (P=0.645) analgesic activity of clonidine (FIG. 18B).

The Table below summarizes the analgesic affects of the various agents.

TABLE 1

| | Morphine | | Oxycodone | |
|---|---|---|---|---|
| Antagonist | Clonidine | BMS182874 | Clonidine | BMS182874 |
| Vehicle | Increase | Increase | Increase | Increase |
| Idazoxan | No effect | No effect | Blocked | Blocked |
| Yohimbine | Blocked | No effect | Blocked | No effect |
| | -α₂- | -α₂- | -α₂- | -α₂-Adrenergic |
| | Adrenergic | Adrenergic | Adrenergic | receptors not |
| | receptors | receptors not | receptors | involved |
| | involved | involved | involved | -I₁- |
| | -I₁- | -I₁- | -I₁- | imidazoline |
| | imidazoline | imidazoline | imidazoline | receptors |
| | receptors not | receptors not | receptors | involved |
| | involved | involved | involved | |

The conclusion that clonidine and BMS182874 act through different mechanisms was further confirmed by the results above, showing that potentiation of morphine and oxycodone analgesia by clonidine was not affected by treatment with BMS182874. Similarly, potentiation of morphine and oxycodone analgesia by BMS182874 was not affected by treatment with clonidine. Furthermore, clonidine produced analgesic effect which is not affected by treatment with BMS182874 (FIG. 18). All these findings indicate that clonidine and BMS182874 potentiate morphine and oxycodone analgesia through different mechanisms.

Additionally, since yohimbine blocked clonidine-induced potentiation of morphine analgesia but idazoxan did not affect clonidine-induced potentiation of morphine analgesia, it can be concluded that α₂ adrenergic receptors are involved in clonidine's analgesic affects, but I₁-imidazoline receptors are not involved in clonidine induced potentiation of morphine analgesia. The discovery herein that α₂ adrenergic agonists and endothelin A antagonists can effectively potentiate the analgesic effects of opioids suggests that treatment with a combination of agents will effectively reduced the amount of opioid necessary to decrease pain in a treated subject. Further, the studies herein demonstrate that α₂ adrenergic agonists and endothelin A antagonists given in combination synergize and produce analgesics effects similar to a high dose of morphone. Thus, compositions comprising a combination of α₂ adrenergic agonists and endothelin A antagonists, or compositions comprising a single agent, but administered in concert, are useful to treat symptoms of pain without risk of tolerance or addition to opiod analgesics.

Numerous modifications and variations in the invention as set forth in the above illustrative examples are expected to occur to those skilled in the art. Consequently only such limitations as appear in the appended claims should be placed on the invention.

### REFERENCES

[1] Adithan, C., Sivagnanam, G., Swain, R., Shashindran, C. H. and Bapna, J. S., Potentiation of clonidine analgesia by amitriptyline, Indian J Exp Biol, 24 (1986) 256-8.
[2] Backonja, M., Beydoun, A., Edwards, K. R., Schwartz, S. L., Fonseca, V., Hes, M., LaMoreaux, L. and Garofalo, E., Gabapentin for the symptomatic treatment of painful neuropathy in patients with diabetes mellitus: a randomized controlled trial, Jama, 280 (1998) 1831-6.
[3] Bennett, G. J., Update on the neurophysiology of pain transmission and modulation: focus on the NMDA-receptor, J Pain Symptom Manage, 19 (2000) S2-6.
[4] Bhalla, S., Ciaccio, N., Wang, Z. J. and Gulati, A., Involvement of endothelin in morphine tolerance in neuroblastoma (SH-SY5Y) cells, Exp Biol Med (Maywood), 231 (2006) 1152-6.
[5] Bhalla, S., Matwyshyn, G. and Gulati, A., Potentiation of morphine analgesia by BQ123, an endothelin antagonist, Peptides, 23 (2002) 1837-45.
[6] Bhalla, S., Matwyshyn, G. and Gulati, A., Endothelin receptor antagonists restore morphine analgesia in morphine tolerant rats, Peptides, 24 (2003) 553-61.
[7] Bhalla, S., Matwyshyn, G. and Gulati, A., Morphine tolerance does not develop in mice treated with endothelin-A receptor antagonists, Brain Res, 1064 (2005) 126-35.
[8] Chan, M. F., Okun, I., Stavros, F. L., Hwang, E., Wolff, M. E. and Balaji, V. N., Identification of a new class of ET_{A} selective endothelin antagonists by pharmacophore directed screening, Biochem Biophys Res Commun, 201 (1994) 228-34.
[9] Constant, I., Gall, O., Gouyet, L., Chauvin, M. and Murat, I., Addition of clonidine or fentanyl to local anaesthetics prolongs the duration of surgical analgesia after single shot caudal block in children, Br J Anaesth, 80 (1998) 294-8.
[10] D'Amour, F. E., Smith, D. L., A method for determining loss of pain sensation, J Pharmacol Exp Ther, 71 (1941) 74-79.
[11] Fromm, G. H., Aumentado, D. and Terrence, C. F., A clinical and experimental investigation of the effects of tizanidine in trigeminal neuralgia, Pain, 53 (1993) 265-71.
[12] Gilron, I., Bailey, J. M., Tu, D., Holden, R. R., Weaver, D. F. and Houlden, R. L., Morphine, gabapentin, or their combination for neuropathic pain, N Engl J Med, 352 (2005) 1324-34.
[13] Gordon, N. C., Heller, P. H. and Levine, J. D., Enhancement of pentazocine analgesia by clonidine, Pain, 48 (1992) 167-9.
[14] Goudas, L. C. and Carr, D. B., Is there a place for intrathecal clonidine for clinical analgesia? Anesth Analg, 83 (1996) 891.
[15] Gulati, A., Evidence for antagonistic activity of endothelin for clonidine induced hypotension and bradycardia, Life Sci, 50 (1992) 153-60.
[16] Gulati, A., Bhalla, S. and Matwyshyn, G., A Novel Combination of Opiates and Endothelin Antagonists to Manage Pain Without Any Tolerance Development, J Cardiovasc Pharmacol, 44 (2004) S129-S131.
[17] Gulati, A., Rebello, S. and Kumar, A., Role of sympathetic nervous system in cardiovascular effects of centrally administered endothelin-1 in rats, Am J Physiol, 273 (1997) H177-86.
[18] Gulati, A. and Srimal, R. C., Endothelin antagonizes the hypotension and potentiates the hypertension induced by clonidine, Eur J Pharmacol, 230 (1993) 293-300.
[19] Hutschala, D., Mascher, H., Schmetterer, L., Klimscha, W., Fleck, T., Eichler, H. G. and Tschemko, E. M., Clonidine added to bupivacaine enhances and prolongs analgesia after brachial plexus block via a local mechanism in healthy volunteers, Eur J Anaesthesiol, 21 (2004) 198-204.
[20] Jamali, S., Monin, S., Begon, C., Dubousset, A. M. and Ecoffey, C., Clonidine in pediatric caudal anesthesia, Anesth Analg, 78 (1994) 663-6.
[21] Knotkova, H. and Pappagallo, M., Adjuvant analgesics, Med Clin North Am, 91 (2007) 113-24.
[22] Kunos, G., Mosqueda-Garcia, R., Mastrianni, J. A. and Abbott, F. V., Endorphinergic mechanism in the central cardiovascular and analgesic effects of clonidine, Can J Physiol Pharmacol, 65 (1987) 1624-32.
[23] Lim, D. Y., Heo, K., Choi, C. H. and Lee, E. W., Influence of endothelin-1 on clonidine-induced cardiovascular effects in anesthetized rabbits, J Cardiovasc Pharmacol, 31 Suppl 1 (1998) S122-5.
[24] Lyons, B., Casey, W., Doherty, P., McHugh, M. and Moore, K. P., Pain relief with low-dose intravenous clonidine in a child with severe burns, Intensive Care Med, 22 (1996) 249-51.
[25] Mandell, G. and Sande, M., Antimicrobial Agents, In Goodman and Gilman's The Pharmacological Basis of Therapeutics, Eighth Edition, Pergamon Press, New York, 1991, 1047-1064 pp.
[26] Matwyshyn, G. A., Bhalla, S. and Gulati, A., Endothelin ETA receptor blockade potentiates morphine analgesia but does not affect gastrointestinal transit in mice, Eur J Pharmacol, 543 (2006) 48-53.
[27] Milne, B., Cervenko, F. W., Jhamandas, K. and Sutak, M., Intrathecal clonidine: analgesia and effect on opiate withdrawal in the rat, Anesthesiology, 62 (1985) 34-8.
[28] Mutafova-Yambolieva, V., Petkov, O., Staneva-Stoytcheva, D. and Lasova, L., Interactions between the effects of endothelin-1, clonidine and yohimbine on electrically-induced contractions in rat vas deferens, Gen Pharmacol, 23 (1992) 529-34.
[29] Nishina, K. and Mikawa, K., Clonidine in paediatric anaesthesia, Curr Opin Anaesthesiol, 15 (2002) 309-16.
[30] Paalzow, G., Development of tolerance to the analgesic effect of clonidine in rats. Cross-tolerance to morphine, Naunyn Schmiedebergs Arch Pharmacol, 304 (1978) 1-4.
[31] Paalzow, L., Analgesia produced by clonidine in mice and rats, J Pharm Pharmacol, 26 (1974) 361-3.
[32] Porchet, H. C., Piletta, P. and Dayer, P., Objective assessment of clonidine analgesia in man and influence of naloxone, Life Sci, 46 (1990) 991-8.
[33] Puppala, B. L., Matwyshyn, G., Bhalla, S. and Gulati, A., Evidence that morphine tolerance may be regulated by endothelin in the neonatal rat, Biol Neonate, 86 (2004) 138-44.
[34] Puppala, B. L., Matwyshyn, G., Bhalla, S. and Gulati, A., Role of Endothelin in Neonatal Morphine Tolerance, J Cardiovasc Pharmacol, 44 (2004) S383-S385.
[35] Rowbotham, M. C., Goli, V., Kunz, N. R. and Lei, D., Venlafaxine extended release in the treatment of painful diabetic neuropathy: a double-blind, placebo-controlled study, Pain, 110 (2004) 697-706.
[36] Saarto, T. and Wiffen, P. J., Antidepressants for neuropathic pain, Cochrane Database Syst Rev (2005) CD005454.
[37] Semenchuk, M. R., Sherman, S. and Davis, B., Double-blind, randomized trial of bupropion SR for the treatment of neuropathic pain, Neurology, 57 (2001) 1583-8.
[38] Spaulding, T. C., Fielding, S., Venafro, J. J. and Lal, H., Antinociceptive activity of clonidine and its potentiation of morphine analgesia, Eur J Pharmacol, 58 (1979) 19-25.
[39] Wang, Y. C., Su, C. F. and Lin, M. T., The site and the mode of analgesic actions exerted by clonidine in monkeys, Exp Neurol, 90 (1985) 479-88.
[40] Ordonez Gallego A, Gonzalez Baron M, Espinosa Arranz E: Oxycodone: A pharmacological and clinical review. Clin Transl Oncol 2007;9:298-307.
[41] Nielsen C K, Ross F B, Lotfipour S, Saini K S, Edwards S R, Smith M T: Oxycodone and morphine have distinctly different pharmacological profiles: Radioligand binding and behavioural studies in two rat models of neuropathic pain. Pain 2007; 132:289-300.
[42] Nozaki C, Kamei J: Involvement of mul-opioid receptor on oxycodone-induced antinociception in diabetic mice. Eur J Pharmacol 2007;560:160-162.
[43] Falk E: Eukodal ein neues narkotikum [eukodal, a new narcotic]. Muenchener Med Wochenschr 1917;64:381-384.
[44] Yoburn B C, Shah S, Chan K, Duttaroy A, Davis T: Supersensitivity to opioid analgesics following chronic opioid antagonist treatment: Relationship to receptor selectivity. Pharmacol Biochem Behav 1995:51:535-539.
[45] Monory K, Greiner E, Sartania N, Sallai L, Pouille Y, Schmidhammer H, Hanoune J, Borsodi A: Opioid binding profiles of new hydrazone, oxime, carbazone and semicarbazone derivatives of 14-alkoxymorphinans. Life Sci 1999;64:2011-2020.
[46] Peckham E M, Traynor J R: Comparison of the antinociceptive response to morphine and morphine-like compounds in male and female sprague-dawley rats. J Pharmacol Exp Ther 2006;316:1195-1201.
[47] Ernsberger P, Giuliano R, Willette R N, Reis D J: Role of imidazole receptors in the vasodepressor response to clonidine analogs in the rostral ventrolateral medulla. J Pharmacol Exp Ther 1990;253:408-418.
[48] Ernsberger P, Damon T H, Graff L M, Schafer S G, Christen M O: Moxonidine, a centrally acting antihypertensive agent, is a selective ligand for i1-imidazoline sites. J Pharmacol Exp Ther 1993;264:172-182.
[49] Hamilton C A, Yakubu M A, Jardine E, Reid J L: Imidazole binding sites in rabbit kidney and forebrain membranes. Journal of autonomic pharmacology 1991;11:277-283.
[50] Chao, H. M. and Osborne, N. N., Topically applied clonidine protects the rat retina from ischaemia/reperfusion by stimulating alpha(2)-adrenoceptors and not by an action on imidazoline receptors, Brain Res, 904 (2001) 126-36.
[51] Costela, J. L., Carlos, R., Zamacona, M. K., Jimenez, R. and Calvo, R., Interaction between propofol and sulfisoxazole in mice an in vivo and in vitro study, Res Commun Mol Pathol Pharmacol, 93 (1996) 89-100.
[52] Syed, H., Safa, R., Chidlow, G. and Osborne, N. N., Sulfisoxazole, an endothelin receptor antagonist, protects retinal neurones from insults of ischemia/reperfusion or lipopolysaccharide, Neurochem Int, 48 (2006) 708-17.
[53] Iqbal, J., Wig, J., Bhardwaj, N. and Dhillon, M. S., Intra-articular clonidine vs. morphine for post-operative analgesia following arthroscopic knee surgery (a comparative evaluation), Knee, 7 (2000) 109-113.
[54] Goyagi, T., Tanaka, M. and Nishikawa, T., Oral clonidine premedication reduces induction dose and prolongs awakening time from propofol-nitrous oxide anesthesia, Can J Anaesth, 46 (1999) 894-6.
[55] Constant, I., Gall, O., Gouyet, L., Chauvin, M. and Murat, I., Addition of clonidine or fentanyl to local anaesthetics prolongs the duration of surgical analgesia after single shot caudal block in children, Br J Anaesth, 80 (1998) 294-8.
[56] Ezri, T., Szmuk, P., Shklar, B., Katz, J. and Geva, D., Oral clonidine premedication does not prolong analgesia after hemiorrhaphy under subarachnoid anesthesia, J Clin Anesth, 10 (1998) 474-81.
[57] Tan, P. H., Chou, A. K., Pemg, J. S., Chung, H. C., Lee, C. C. and Mok, M. S., Comparison of epidural butorphanol plus clonidine with butorphanol alone for postoperative pain relief, Acta Anaesthesiol Sin, 35 (1997) 91-6.
[58] Rockemann, M. G., Seeling, W., Duschek, S., Reinelt, H., Steffen, P. and Georgieff, M., Epidural bolus clonidine/morphine versus epidural patient-controlled bupivacaine/sufentanil: quality of postoperative analgesia and cost-identification analysis, Anesth Analg, 85 (1997) 864-9.
[59] Buggy, D. J. and MacDowell, C., Extradural analgesia with clonidine and fentanyl compared with 0.25% bupivacaine in the first stage of labour, Br J Anaesth, 76 (1996) 319-21.
[60] Lena, P., Balarac, N., Arnulf, J. J., Teboul, J. and Bonnet, F., Intrathecal morphine and clonidine for coronary artery bypass grafting, Br J Anaesth, 90 (2003) 300-3.
[61] Bonhomme, V., Doll, A., Dewandre, P. Y., Brichant, J. F., Ghassempour, K. and Hans, P., Epidural administration of low-dose morphine combined with clonidine for postoperative analgesia after lumbar disc surgery, J Neurosurg Anesthesiol, 14 (2002) 1-6.
[62] Jeffs, S. A., Hall, J. E. and Morris, S., Comparison of morphine alone with morphine plus clonidine for postoperative patient-controlled analgesia, Br J Anaesth, 89 (2002) 424-7.
[63] Goyagi, T. and Nishikawa, T., Oral clonidine premedication enhances the quality of postoperative analgesia by intrathecal morphine, Anesth Analg, 82 (1996) 1192-6.
[64] Yanagidate, F., Hamaya, Y. and Dohi, S., Clonidine premedication reduces maternal requirement for intravenous morphine after cesarean delivery without affecting newborn's outcome, Reg Anesth Pain Med, 26 (2001) 461-7.
[65] Park, J., Forrest, J., Kolesar, R., Bhola, D., Beattie, S. and Chu, C., Oral clonidine reduces postoperative PCA morphine requirements, Can J Anaesth, 43 (1996) 900-6.
[66] van Essen, E. J., Bovill, J. G., Ploeger, E. J. and Houben, J. J., Pharmacokinetics of clonidine after epidural administration in surgical patients. Lack of correlation between plasma concentration and analgesia and blood pressure changes, Acta Anaesthesiol Scand, 36 (1992) 300-4).
[67] Smith, B. D., Baudendistel, L. J., Gibbons, J. J. and Schweiss, J. F., A comparison of two epidural alpha 2-agonists, guanfacine and clonidine, in regard to duration of antinociception, and ventilatory and hemodynamic effects in goats, Anesth Analg, 74 (1992) 712-8.)
[68] Capogna, G., Celleno, D., Zangrillo, A., Costantino, P. and Foresta, S., Addition of clonidine to epidural morphine enhances postoperative analgesia after cesarean delivery, Reg Anesth, 20 (1995) 57-61.
[69] Benhamou, D., Narchi, P., Hamza, J., Marx, M., Peyrol, M. T. and Sembeil, F., Addition of oral clonidine to postoperative patient-controlled analgesia with i.v. morphine, Br J Anaesth, 72 (1994) 537-40.
[70] Carabine, U. A., Milligan, K. R., Mulholland, D. and Moore, J., Extradural clonidine infusions for analgesia after total hip replacement, Br J Anaesth, 68 (1992) 338-43.

What is claimed:

1. A method of treating or preventing pain comprising administering to a mammal in need thereof a therapeutically effective amount of an alpha-2 (α₂) adrenergic receptor agonist and a therapeutically effective amount of an endothelin receptor antagonist.

2. The method of claim 1 wherein the α₂ adrenergic agonist is clonidine.

3. The method of claim 1 wherein the endothelin receptor antagonist is an endothelin receptor A (ET_{A}) antagonist.

4. The method of claim 3 wherein the ET_{A} antagonist is selected from the group consisting of sulfosoxazole, atrasentan, tezosentan, bosentan, sitaxsentan, enrasentan, BMS 207940, BMS 193884, BMS 182874, J 104132, VML 588/Ro 61 1790, T-0115, TAK 044, BQ 788, TBC2576, TBC3214, PD180988, ABT 546, SB247083, RPR118031A and BQ123.

5. The method of claim 4 wherein the ET_{A} antagonist is sulfisoxazole.

6. The method of claim 1 wherein the α₂ adrenergic agonist and the endothelin antagonist are administered in a single composition.

7. The method of claim 1 wherein the α₂ adrenergic agonist and the endothelin receptor antagonist are administered in separate compositions.

8. The method of claim 7 wherein the compositions are administered concurrently.

9. The method of claim 7 wherein the compositions are administered separately.

10. The method of any one of claims 7 through 9 wherein the compositions further comprise a pharmaceutical carrier or excipient.

11. The method of claim 1 wherein the α₂ adrenergic agonist and the endothelin receptor antagonist are administered orally, buccally, via inhalation, sublingually, rectally, vaginally, intracisternally, intraarticularly, transurethrally, nasally, percutaneously, intravenously, intramuscularly, or subcutaneously.

12. The method of claim 2 wherein the clonidine is administered in a dose range from 10 μg to about 300 μg.

13. The method of claim 4 wherein the sulfisoxazole is administered in a dose range from 0.1 g to about 3 g.

14. The method of claim 1 wherein the ratio of α₂ adrenergic agonist administered to endothelin receptor antagonist administered is in the range of 1:500 to 1:50,000, 1:500 to 1:20,000,1:500 to 1:10,000, 1:500 to 1:5,000, 1:500 to 1:2,500, 1:100 to 1:1000, or 1:100 to 1:500.

15. A method of treating or preventing pain comprising administering to a subject a synergistic combination of one or more alpha-2 (α₂) adrenergic agonist and one or more endothelin receptor antagonist.

16. The method of claim 1 or 15 wherein the agonist and antagonist molecules of the combination are administered sequentially within about a 24-hour period or are administered concurrently.

17. A method of treating or preventing pain comprising administering to a mammal in need thereof a therapeutically effective amount of an opiate analgesic, and a therapeutically effective amount of a composition comprising one or one or more alpha-2 (α₂) adrenergic agonist.

18. A method of treating or preventing pain comprising administering to a mammal in need thereof a therapeutically effective amount of an opiate analgesic, a therapeutically effective amount of a composition comprising one or one or more alpha-2 (α₂) adrenergic agonist, and a therapeutically effective amount of a composition comprising one or more endothelin receptor antagonist.

19. A method of treating or preventing pain comprising administering to a mammal in need thereof a therapeutically effective amount of an opiate analgesic, and a therapeutically effective amount of a composition comprising one or one or more alpha-2 (α₂) adrenergic agonist and one or more an endothelin receptor antagonist.

20. The method of any one of claims 17 to 19 wherein the α₂ adrenergic agonist is clonidine.

21. The method of any one of claims 17 to 19 wherein the endothelin receptor antagonist is an endothelin receptor A (ET_{A}) antagonist.

22. The method of any one of claims 17 to 19 wherein the ET_{A} antagonist is selected from the group consisting of sulfosoxazole, atrasentan, tezosentan, bosentan, sitaxsentan, enrasentan, BMS207940, BMS193884, BMS182874, J 104132, VML 588/Ro 61 1790, T-0115, TAK 044, BQ 788, TBC2576, TBC3214, PD180988, ABT 546, SB247083, RPR118031A and BQ123.

23. The method of any one of claims 17 to 19 wherein the opiate analgesic is selected from the group consisting of morphine, morphine sulfate, codeine, diacetylmorphine; dextromethorphan, hydrocodone, hydromorphone, hydromorphone, levorphanol, oxymorphone, oxycodone, levallorphan and salts thereof.

24. A composition for treating or preventing pain comprising a synergistic combination of one or more alpha-2 (α₂) adrenergic agonist and one or more endothelin receptor antagonist.

25. The composition of claim 17, comprising a low dose of the α₂ adrenergic agonist and a low dose of the endothelin receptor antagonist.

26. The composition of claim 18 wherein the α₂ adrenergic agonist is clonidine and the endothelin receptor antagonist is sulfisoxazole.

27. The composition of claim 19, wherein the clonidine in the composition is in a range of 10 μg to about 300 μg and the sulfisoxazole in the composition is in a range of about 0.1 g to about 3 g.

28. The composition of any one of claims 25 to 27 further comprising a pharmaceutically acceptable carrier.

29. Use of a composition comprising an alpha-2 (α₂) adrenergic agonist and an endothelin antagonist for the manufacture of a medicament for treating pain in a subject.

30. The method of any one of claims 1 to 23 or use of claim 29 wherein the subject is a mammal.

31. The method of claim 30 wherein the subject is human.

32. The method of any one of claims 1 to 23 or use of claim 29 wherein the pain is chronic pain

33. The method of any one of claims 1-23 or use of claim 29 wherein the pain is acute pain.

34. An article of manufacture comprising an alpha-2 (α₂) adrenergic agonist and an endothelin receptor antagonist and a label indicating a method according to any one of claims 1 or 15 to 19.

35. A kit for treating or preventing pain comprising:
(a) the composition of any one of claims 24 to 28; and
(b) a protocol for using the kit to treat pain.

36. The kit of claim 35 further comprising an opiate analgesic.
